(19) United States
(12) Patent Application Publication
(10) Pub. No.: US 2023/0133655 A1
(43) Pub. Date: May 4, 2023

(54) Materials and Methods for Biosorption of Micropollutants from Aqueous Solutions

(71) Applicant: Massachusetts Institute of Technology, Cambridge, MA (US)

(72) Inventors: Patritsia Maria Stathatou, Boston, MA (US); Filippos Tourlomousis, Cambridge, MA (US); Christos Edouardos Athanasiou, Boston, MA (US); Andreas Mershin, Arlington, MA (US); Neil A. Gershenfeld, Cambridge, MA (US)

(73) Assignee: Massachusetts Institute of Technology, Cambridge, MA (US)

(21) Appl. No.: 17/941,016

(22) Filed: Sep. 8, 2022

Related U.S. Application Data
(60) Provisional application No. 63/241,968, filed on Sep. 8, 2021.

(51) Int. Cl. C02F 3/34; C12N 1/18; C02F 1/66

(52) U.S. Cl. CPC C02F 3/347; C12N 1/18; C02F 1/66; C02F 2101/30

(57) ABSTRACT

A method for removing organic or inorganic micropollutants from aqueous solutions includes adding a biomass of biomaterial to the solutions, the biomaterial being an active or inactive biological organism, such as a yeast, having affinity for biosorption (adsorption by the biomass)of an organic or inorganic micropollutant present in the solution at, or below, a parts-per-billion concentration, and controlling the pH and temperature of the solution, as well as contact time and agitation, to be within a range suitable for biosorption of the at micropollutant by the biomaterial. The amount of biomass added to the solution may be calculated according to the amount of solution, the concentration of the micropollutant in the solution, and the total amount of the micropollutant that can be biosorbed by a particular quantity of the inactive or active biomaterial. In a preferred embodiment, the biomaterial is obtained from Saccharomyces cerevisiae.

### RELATED APPLICATIONS

This application claims the benefit of U.S. Provisional Application Ser. No. 63/241,968, filed Sep. 8, 2021, the entire disclosure of which is herein incorporated by reference.

### FIELD OF THE TECHNOLOGY

The present invention relates to environmental remediation and, in particular, to a biomaterial for removing recalcitrant, non-biodegradable micropollutants from aqueous solutions.

### BACKGROUND

Micropollutants are biological or chemical contaminants that can be found in groundwater and surface water bodies in trace concentrations (at microgram per liter level and below). These contaminants include a myriad of natural and synthetic organic compounds and are labeled as “emerging” due to the fact that they have traditionally been unmonitored or unregulated in environmental samples while causing significant damages to human health and ecosystems. The manufacturing, use, and subsequent disposal of these substances, including, but not limited to, pharmaceuticals, personal care products, industrial chemicals (including per- and polyfluoroalkyl substances (PEAS)), cleaning detergents, steroid hormones, and pesticides, has led to their detection in treated water and in wastewater effluents. Similarly, inorganic micropollutants, i.e. heavy metals, such as lead (Pb), arsenic (As), cadmium (Cd), and chromium (Cr), have been found in drinking water across the globe. In addition, in certain instances, chemicals are added to source water on purpose in order to render the final drinking water product safe for consumption, but in the process create undesired chemicals, namely disinfection by-products. Due to the potential of all of these contaminants (and many more) to cause health, economic, and environmental impacts, they have become an issue of global concern [Cornell University, New York State Water Resources Institute, 2021: https://wri.cals.cornell.eduiresearch-topics/micropollutants-emerging-contminants/].

Heavy metals, e.g. cadmium, arsenic, mercury, nickel, and lead, can occur in water bodies due to both natural and anthropogenic activities, such as, but not limited to, mining, burning of fossil fuels, and various manufacturing processes [Rehman, K., Fatima, F., Waheed, I. & Akash, M. S. H., “Prevalence of exposure of heavy metals and their impact on health consequences”, J. Cell. Biochem., 119, 157-184 (2018)]. There is no unanimous definition of what constitutes a heavy metal, but the most commonly used defining factor is a density greater than 5 g/cm³. Electronic waste (e-waste) discharge and mining are the most dominant anthropogenic activities responsible for heavy metal contamination of water resources. Acid mine drainage (AMD), i.e., leakage of highly acidic water rich in metals, is a global environmental threat. In the United States (US) alone, AMD is the main source of water pollution, impacting currently over 20,000 km of streams, deriving from the 13,000 active and the 500,000 abandoned mines, which continue generating AMD for centuries after their closure.

Heavy metals are highly water-soluble. Unlike organic pollutants, which can eventually be destroyed in most cases by chemical and physical means, heavy metals are non-biodegradable, tending to persist indefinitely when released into the environment, circulating and accumulating throughout food chains [Volesky, B., “Advances in biosorption of metals: Selection of biomass types”, FEMS Microbiol. Rev., 14, 291-302 (1994)]. Traces of heavy metals found in water resources pose a global threat. Similar to some microplastic pollutants, heavy metals are non-biodegradable and toxic; hence, their accumulation is hazardous for living organisms and human health, even at trace concentrations. Conventional treatment processes fail to remove toxic heavy metals, such as lead, from drinking water in a resource-efficient manner when their initial concentrations are low.

Lead (Pb), in particular, is one of the most widely used, and at the same time one of the most toxic, heavy metals, with deleterious effects on organs and tissues of the human body, even at trace concentrations, in particular affecting the kidneys and nervous system of young children [Rehman, K., Fatima, F., Waheed, I. & Akash, M. S. H., “Prevalence of exposure of heavy metals and their impact on health consequences”, J. Cell. Biochem., 119, 157-184 (2018); Wani, A. L., Ara, A. & Usmani, J. A., “Lead toxicity: a review.”, Interdiscip. Toxicol. 8, 55-64 (2015); Casas, J. S. & Sordo, J., “Lead: Chemistry, Analytical Aspects, Environmental Impact and Health Effects”, Elsevier (2006); Rubin, R. & Strayer, “Rubin's pathology: Clinicopathologic Foundations of Medicine”, Lippincot Williams & Wilkins (2008); Bressler, J. P. & Goldstein, G. W., “Mechanisms of lead neurotoxicity”, Biochem. Pharmacol. 41, 479-484 (1991); US EPA, “EPA Proposes Updates to Lead and Copper Rule to Better Protect Children and At-Risk Communities”, https://www.epa.govinewsreleases/epa-proposes-updates-lead-and-copper-rule-better-protect-children-and-risk-0 (2019); Barakat, M. A. “New trends in removing heavy metals from industrial wastewater”, Arab. J. Chem. 4, 361-377 (2011); El-Naggar, N. E.-A., Hamouda, R. A., Mousa, I. E., Abdel-Hamid, M. S. & Rabei, N. H., “Biosorption optimization, characterization, immobilization and application of Gelidium amansii biomass for complete Pb2+ removal from aqueous solutions”, Sci. Rep. 8, 13456 (2018)].

The greatest use for Pb today is in Pb-acid batteries, but it is also used for construction purposes, cable sheathing, radiation shielding, synthesis of oxides for paints and pigments, and for other paint additives [Rehman, K., Fatima, F., Waheed, I. & Akash, M. S. H., “Prevalence of exposure of heavy metals and their impact on health consequences”, J. Cell. Biochem., 119, 157-184 (2018); Casas, J. S. & Sordo, J., “Lead: Chemistry, Analytical Aspects, Environmental Impact and Health Effects”, Elsevier (2006)]. Its production increased by about 20% during the last decade, reaching around 11.7 million tons globally in 2020 [Statista, “World production of lead from 2006 to 2020”, https://www.statista.com/statistics/264872/world-production-of-lead-metal/(2021)]. Pb can enter drinking water, either due to inadequate water treatment or due to its release from Pb-containing components of water distribution systems and plumbing materials, such as Pb pipes, solder, and brass fittings in faucets and fixtures, when a chemical reaction occurs [CDC, “Sources of Lead”, Childhood Lead Poisoning Prevention https://www.cdc.gov/nceh/lead/prevention/sources.htm (2020); Santucci, R. J. & Scully, J. R., “The pervasive threat of lead (Pb) in drinking water: Unmasking and pursuing scientific factors that govern lead release”, Proc. Natl. Acad. Sci. 117, 23211-23218 (2020); Sharma, P. R. et al., “Lead removal from water using carboxycellulose nanofibers prepared by nitro-oxidation method”, Cellulose 25, 1961-1973 (2018)]. Pb is also present in, and can be leached from, Polyvinyl chloride (PVC) pipes [WHO, “Lead in Drinking-water”, Background document for development of WHO Guidelines for Drinking-water Quality, https://www.who.int/water_sanitation_health/dwq/chemicals/lead.pdf (2011)].

Pb contamination of drinking water is a serious global issue. After serious Pb-contamination incidents in the western world (e.g. Glasgow—Scotland, Washington, D.C., public schools in Los Angeles, Baltimore, and Seattle [Santucci, R. J. & Scully, J. R., “The pervasive threat of lead (Pb) in drinking water: Unmasking and pursuing scientific factors that govern lead release”, Proc. Natl. Acad. Sci. 117, 23211-23218 (2020); WHO, “Lead in Drinking-water”, Background document for development of WHO Guidelines for Drinking-water Quality, (2011)], with the most recent one the water crisis in the city of Flint, Mich., USA in 2014), relevant standards and regulations are being revisited and limits of Pb in drinking water are becoming more stringent. In 2018, the European Commission proposed halving Pb limits, from a maximum of 10 parts per billion (ppb-μg/L) down to 5 ppb over a transitional period of 10 years [Aquatech, “Lead levels to be halved under revised Drinking Water Directive”, https://www.aquatechtrade.com/news/water-treatment/lead-levels-to-be-halved-under-revised-drinking-water-directive/(2018)]. In 2020, the United States Environmental Protection Agency (US EPA), the U.S. Centers for Disease Control and Prevention, and Health Canada have determined that no level of Pb in drinking water is considered safe [US EPA, “EPA Proposes Updates to Lead and Copper Rule to Better Protect Children and At-Risk Communities” (2019); NSF International, “Revisions to drinking water standard tighten lead leaching allowance for plumbing products”, https://phys.org/news/2020-09-standard-tighten-leaching-plumbing-products.html?utm_source=nwletter&utm_medium=email&utm_campaign=daily-nwletter (2020)]. Pb is unsafe even at trace concentrations, and therefore there is an urgent need to eliminate, rather than just minimize, its occurrence in wastewater prior to discharge, as well as in drinking water prior to consumption, in a systematic manner.

Mean Pb concentrations in global surface water bodies are more than 10 times greater than they were in the 1970s, ranging from around 10 ppb in Europe to hundreds of ppb in South America [Zhou, Q. et al., “Total concentrations and sources of heavy metal pollution in global river and lake water bodies from 1972 to 2017”, Glob. Ecol. Conserv. 22, e00925 (2020)]. Conventional water and wastewater treatment methods for removing Pb from aqueous solutions, such as chemical precipitation, ion exchange, chemical oxidation or reduction, membrane filtration, and electrochemical deposition, either fail to completely remove trace Pb amounts or result in significant energy consumption, increased operational and maintenance costs, or generation of toxic by-products to do so [Klein, R., Tischler, J. S., Willing, M. & Schlomann, M., “Bioremediation of Mine Water”, Geobiotechnology I, vol. 141, 109-172, Springer Berlin Heidelberg, 2013); Johnson, D. B. & Hallberg, K. B., “Acid mine drainage remediation options: a review”, Sci. Total Environ. 338, 3-14 (2005); Sun, G. L., Reynolds, Erin, E. & Belcher, A. M., “Using yeast to sustainably remediate and extract heavy metals from waste waters”, Nat. Sustain. 3, 303-311 (2020); Tian, H., Alkhadra, M. A., Conforti, K. M. & Bazant, M. Z., “Continuous and Selective Removal of Lead from Drinking Water by Shock Electrodialysis”, ACS EST Water acsestwater.1c00234 https://doi.org/10.1021/acsestwater.1c00234 (2021)].

More specifically, chemical precipitation, which is the most widely used method for heavy metal removal to-date [Barakat, M. A. “New trends in removing heavy metals from industrial wastewater”, Arab. J. Chem. 4, 361-377 (2011); Sun, G. L., Reynolds, Erin. E. & Belcher, A. M.' “Using yeast to sustainably remediate and extract heavy metals from waste waters”, Nat. Sustain. 3, 303-311 (2020); Farooq, U., Kozinski, J. A., Khan, M. A. & Athar, M., “Biosorption of heavy metal ions using wheat based biosorbents—A review of the recent literature”, Bioresour. Technol. 101, 5043-5053 (2010)], needs significant amounts of chemicals and produces a concentrated toxic sediment that is at least 5 times greater than the initial amount of the target heavy metal in wastewater, requiring further treatment and special waste management practices [Fabiani, C., “Metal removal from aqueous wastes by means of membrane hybrid processes”, Recents Progress an Genie des Procedes, Membrane Processes Water Treatment-Pervaporation, 211, Lavoisier Press (1992)]. In addition, this method is not cost-effective for metal concentrations below 100 parts per million (ppm or mg/l) [Haluk Ceribasi, I. & Yetis, U., “Biosorption of Ni(ii) and Pb(ii) by Phanerochaete chrysosporium from a binary metal system—kinetics”, Water SA 27,15-20 (2004)]. Ion exchange, which is also a widely applied process for Pb removal, is non-selective, prone to fouling, and an expensive treatment method [Barakat, M. A. “New trends in removing heavy metals from industrial wastewater”, Arab. J. Chem. 4,361-377 (2011); Farooq, U., Kozinski, J. A., Khan, M. A. & Athar, M., “Biosorption of heavy metal ions using wheat based biosorbents—A review of the recent literature”, Bioresour. Technol. 101, 5043-5053 (2010)]. Membrane filtration also involves high operational costs and is prone to fouling. While the cheaper of these processes are becoming inadequate with progressively stricter regulatory effluent limits, more effective methods are prohibitively costly [Volesky, B., “Advances in biosorption of metals: Selection of biomass types”, FEMS Microbiol. Rev., 14,291-302 (1994)].

A significant amount of research is being conducted on cheaper, more effective, and less wasteful processes for removal of heavy metals, microplastics, and other inorganic pollutants, particularly by engineered microorganisms and designed substrates. Adsorption, a mass transfer process by which heavy metal ions or other dissolved particles are transferred from the liquid phase to the surface of a solid and are bound by physical and/or chemical interactions, is considered a competitive alternative to conventional treatment processes and as a solution for removal of difficult-to-chelate pollutants. An expansive range of both natural and synthetic materials have been studied as potential metal-binding adsorbents, including minerals, clays, and industrial byproducts such as fly ash, iron slags, and titanium dioxide. Particularly, adsorbents of biological origin—biomaterials, such microorganisms, plants, and agricultural waste—have been gaining attention as they can offer promising, low-cost, and environmentally friendly substrates for heavy metal and other pollutant removal.

Sorption and/or complexation of dissolved heavy metal ions as a function of the chemical activity of inactive biomass is called biosorption [Volesky, B., “Advances in biosorption of metals: Selection of biomass types”, FEMS Microbiol. Rev., 14,291-302 (1994); Tsezos, M., “Biosorption: A Mechanistic Approach”, Geobiotechnology I: Metal-related Issues (eds. Schippers, A., Glombitza, F. & Sand, W.) 173-209, Springer-Verlag (2014)] and the same term is used herein to cover adsorption processes. Biosorption of heavy metals using a wide variety of biomaterials has been studied at the parts per million (ppm) contaminants concentration for decades. However, the mechanisms of metal biosorption are complicated and still not fully understood [Wang, J. & Chen, C., “Biosorption of heavy metals by Saccharomyces cerevisiae: A review”, Biotechnol. Adv. 24,427-451 (2006)], with only a small subset of the work reported in the literature devoted to their systematic and explicit elucidation. Most of the times the reported biosorptive uptake capacities do not refer to the very important prevailing experimental conditions, such as the rigorous definition of the biosorbing species, the solution equilibrium pH, the sorbate speciation and residual concentration, or the equilibrium attainment contact time [Tsezos, M., “Biosorption: A Mechanistic Approach”, Geobiotechnology I: Metal-related Issues, 173-209, Springer-Verlag (2014)]. The initial metal ion concentration has a strong effect on the biosorption capacity, while decreasing initial concentrations are linked with decreasing uptake capacities from the biosorbents, if the amount of microbial biomass is kept unchanged [Wang, J. & Chen, C., “Biosorption of heavy metals by Saccharomyces cerevisiae: A review”, Biotechnol. Adv. 24,427-451 (2006)].

Biosorption of heavy metals with initial concentrations at the ppb scale and below poses a challenging and currently unexplored area, as the driving forces decrease, and the underlying adsorption mechanisms are unknown. To date, it appears that inactive yeast biomass, and in particular lyophilized Saccharomyces boulardii (Saccharomyces cerevisiae sp.) cells, have never before been used to remove heavy metals from aqueous solutions at trace concentrations.

### SUMMARY

The present invention is a sustainable, biological method that uses an abundant, low-value or waste biomaterial for removing recalcitrant, non-biodegradable micropollutants of emerging concern, including, but not limited to, heavy metals, pharmaceuticals and personal care products, cleaning detergents, steroid hormones, pesticides, industrial chemicals, such as per- and polyfluoroalkyl substances (PFAS), and microplastics from aqueous solutions, including, but not limited to, drinking water and wastewater. In particular, the present invention has been used for removing inorganic micropollutants from aqueous solutions. Among other uses, the present invention has particular application in the fields of water purification, wastewater treatment, and environmental remediation in terrestrial and extraterrestrial environments.

In one particular embodiment, the invention comprises the use of inactive yeast biomass, particularly lyophilized Saccharomyces boulardii (Saccharomyces cerevisiae sp.) cells, to remove lead (Pb) from aqueous solutions at trace concentrations. These yeast cells are cultivated, washed and lyophilized under specific conditions to produce a yeast biomass powder. Using this biomass powder, lead ions (Pb⁺²) at the challenging micropollutants levels have been effectively removed from aqueous solutions with initial Pb concentrations of 20-1000 ppb. By using the yeast Saccharomyces cerevisiae, trace lead is effectively removed from water via a rapid mass transfer process, called biosorption, achieving an uptake of up to 12 mg lead per gram of biomass in solutions with initial lead concentrations below 1 part per million. Through spectroscopic analyses, it was found that the yeast cell wall plays a crucial role in this process, with its mannoproteins and β-glucans being the key potential lead adsorbents. Furthermore, by employing nanomechanical characterization in the yeast biomass, it was discovered that biosorption is linked to an increase in cell wall stiffness. These findings open new opportunities for using environmentally friendly and abundant biomaterials for advanced water treatment targeting emerging contaminants.

In one aspect, the present invention is a method that employs a biomaterial to remove recalcitrant, non-biodegradable inorganic micropollutants from aqueous solutions such as, but not limited to, heavy metals at trace concentrations (part per billion scale—ppb) or other concentrations or in admixtures. In another aspect, the present invention is an apparatus that includes a biomaterial for removing recalcitrant, non-biodegradable inorganic micropollutants from aqueous solutions.

In one particular embodiment, the invention is a method for removing trace amounts of organic or inorganic micropollutants from an aqueous solution, comprising adding a biomass of biomaterial to the solution, the biomaterial comprising at least one biological organism selected for its affinity for biosorption of at least one micropollutant that is present in the solution in a concentration level of at or below parts-per-billion, controlling the pH and temperature of the aqueous solution to be within pH and temperature ranges suitable for biosorption of the at least one micropollutant from the solution by the biomaterial, and maintaining agitation and contact between the biomass and the solution for a time suitable for biosorption of the at least one micropollutant from the solution by the biomaterial. The amount of biomass added to the solution may be calculated according to the amount of solution, the concentration of the micropollutant in the solution, and the total amount of the micropollutant that can be biosorbed by a particular quantity of the biomaterial. The biomaterial may be active or inactive. In a preferred embodiment, the biomaterial is at least one yeast, which may be Saccharomyces cerevisiae. In a preferred embodiment, the pH range is pH3 to pH7. The pH of the solution may be adjusted to account for a change in pH of the solution when the biomass is added to the solution.

In another particular embodiment, the invention is a biomaterial for removing trace amounts of an organic or inorganic micropollutant from an aqueous solution, comprising a biomass of at least one biological organism, the biological organism being selected for its particular affinity for biosorption of an inorganic micropollutant that is present in the solution in a concentration level of at or below parts-per-billion, the biological organism having been incubated, harvested, washed, and lyophilized under conditions designed to maximize the ability of the biological organism to biosorb the micropollutant, wherein the amount of biological organism in the biomass is calculated according to the amount of solution, the concentration of the inorganic micropollutant in the solution, and the total amount of the micropollutant that can be biosorbed by a particular quantity of the biological organism at a prespecified pH, temperature, agitation, and contact time. The biological organism may be active or inactive. In a preferred embodiment, the biological organism may be at least one yeast, and in particular may be Saccharomyces cerevisiae. The biological organism may be selected for its ability to biosorb the inorganic micropollutant at the pH of the aqueous solution or within a pH range to which the aqueous solution can be adjusted.

### BRIEF DESCRIPTION OF THE DRAWINGS

Other aspects, advantages and novel features of the invention will become more apparent from the following detailed description of the invention when considered in conjunction with the accompanying drawings, wherein:

FIG. 1 is a measured growth curve of the yeast S. cerevisiae strain used (S. boulardii) used.

FIG. 2 is a plot of results from High-Performance Liquid Chromatography (HPLC) performed to identify the number of washes with ultrapure water required to remove medium residues and metabolites from harvested yeast cells before being used in biosorption experiments for pure culture medium, supernatant of harvested liquid yeast culture, supernatant after the first wash of yeast cells with ultrapure water, and supernatant after the second wash of yeast cells with ultrapure water.

FIG. 3A depicts yeast powder after lyophilization and FIG. 3B depicts scanning electron microscopy (SEM) imaging of freeze-dried yeast cells.

FIG. 4 depicts the main steps of kinetic and equilibrium experiments involving the addition of freeze-dried yeast cells in Pb-containing aqueous solutions, the adsorption of Pb ions, and the separation of biomass and supernatant after the required contact time via centrifugation for further analyses, according to one aspect of the invention.

FIG. 5 is a graph of the distribution of lead(II) nitrate [Pb(NO₃)₂] hydrolysis products at 25° C. and 1 uM Pb(NO₃)₂.

FIG. 6 is a graph showing the increase in solution pH due to yeast biomass addition.

FIG. 7 is a graph depicting the effect of initial pH of solution on Pb²⁺ uptake for 5 mg of yeast biomass and C₀ of 100 ppb Pb²⁺, with the final pH of solutions after biopsorption also reported for each case.

FIG. 8 is a plot of results from kinetic experiments at progressively long increments of contact time for 5 mg yeast biomass with C₀ of 100 ppb Pb²⁺, indicating the rapid biosorption processes.

FIG. 9 is a plot of the adsorption isotherm at 25° C., following the Langmuir adsorption isotherm model.

FIG. 10 is a plot of Pb²⁺ percentage removal versus the initial Pb²⁺ concentration, C₀.

FIGS. 11A and 11B are SEM images depicting an overview of control yeast cells (FIG. 11A) and yeast cells after Pb²⁺ biosorption (FIG. 11B) with C₀ being 100 ppb.

FIGS. 12A and 12B are magnified SEM images showing individual control yeast cells (FIG. 12A) and individual yeast cells after Pb²⁺ biosorption (FIG. 12B) with C₀ being 100 ppb.

FIGS. 13A and 13B are transmission electron microscopy (TEM) images of individual control yeast cells (FIG. 13A) and individual yeast cells after Pb²⁺ biosorption (FIG. 13B) with C₀ being 100 ppb.

FIGS. 14A and 14B are magnified TEM images of control yeast cell walls (FIG. 14A) and yeast cell walls after Pb biosorption (FIG. 14B).

FIG. 15 is a graph depicting the full attenuated-total-reflectance enhanced Fourier transformed infrared spectroscopy (ATR-FTIR) spectrum of yeast cells.

FIGS. 16A-C are graphs depicting the results of X-ray photoelectron spectroscopy (XPS) analysis of yeast cells, wherein FIG. 16A depicts results for control cells that have not been exposed to Pb, FIG. 16B depicts results for yeast cells after Pb²⁺ biosorption (C₀: 100 ppb Pb²⁺), and FIG. 16C depicts results for yeast cells after Pb²⁺ biosorption (C₀: 1000 ppb Pb²⁺).

FIGS. 17A and 17B are a TEM image of a Saccharomyces cerevisiae cell (FIG. 17A) and a schematic of Saccharomyces cerevisiae cell wall structure (FIG. 17B).

FIG. 18 is a graph illustrating chitin's contribution to Pb²⁺ adsorption.

FIG. 19 is a graph depicting results from the nanomechanical characterization of yeast biomass by assessing the yeast cell stiffness via Atomic Force Microscopy (AFM).

### DETAILED DESCRIPTION

In one embodiment of the present invention, a biomaterial, such as, but not limited to, yeast powder, is used for removing recalcitrant, non-biodegradable micropollutants such as, but not limited to, heavy metals at trace concentrations (part per billion scale—ppb) or other concentrations, or in admixtures, from aqueous solutions.

As used herein, “Aqueous solutions” means water-based solutions of varying viscosity including, but not limited to, municipal and other wastewater, potable water, agricultural and industrial effluents, acid mine drainage, and sludge in terrestrial and extraterrestrial environments.

As used herein, “Micropollutants” means water soluble organic and/or inorganic contaminants, both natural or synthetic, including, but not limited to, pharmaceuticals and their metabolic products (such as, but not limited to, sulfamethazine and carbamazepine), industrial chemicals (such as, but not limited to, 1H-benzotriazole, 4-methyl-1H-benzotriazole, and perfluoroalkyl substances—PFAS), personal health products and cosmetics (such as, but not limited to, triclosan), antibiotics (such as, but not limited to, ciprofloxacin and doxycycline), and heavy and other metals (such as, but not limited to, cadmium, arsenic, mercury, nickel, and lead), that are present in water bodies and other aqueous solutions at concentrations ranging from trace levels (at or below micrograms per liter) up to their respective solubility limits, posing serious threats to ecosystems and human health [Rogowska, J., Cieszynska-Semenowicz, M., Ratajczyk, W. et al. Micropollutants in treated wastewater. Ambio 49, 487-503 (2020)]. Although microplastics, i.e. small plastic pieces less than five millimeters long which can be harmful to ocean and aquatic life [National Oceanic and Atmospheric Administration, National Ocean Service, “What are microplastics?”, https://oceanservice.noaa.gov/facts/microplastics.html] are not water soluble and not defined as micropollutants, they have been recognized as transport vectors for micropollutants in water bodies, as micropollutants are being adsorbed on microplastic surfaces. Hence, this definition specifically includes the contaminants formed by the interaction of different organic or inorganic micropollutants with microplastics as well [Ateia, M., Zheng, T., Calace, S., Tharayil, N., Pilla, S. and Karanfil, T., “Sorption behavior of real microplastics (MPs): Insights for organic micropollutants adsorption on a large set of well-characterized MPs”, Science of the Total Environment 720, 137634, (2020)].

As used herein, “Directed adaptation” means techniques that induce phenotypic changes reflected in the genetic background, such as, but not limited to, directed evolution techniques such as, but not limited to, MACE, PACE, directed adaptation via any of the methods known such as MACE, PACE, and the various strategies employed for Evolthon [Kaminski Strauss S, Schirman D, Jona G, Brooks A N, Kunjapur A M, et al., “Evolthon: A community endeavor to evolve lab evolution”, PLOS Biology 17(3): e3000182 (2019)], such as, but not limited to, directed exaptation as disclosed in U.S. patent application Ser. No. 16/133,600 (Mershin et al., Sep. 17, 2018).

This invention is the subject of Stathatou, Patritsia M., Athanasiou, C. E., Tsezos, M., Goss, J. W., Blackburn, L. C., Tourlomousis, F., Mershin, A., Sheldon, B. W., Padture, N. P., Darling, E. M., Gao, H., and Gershenfeld, N. “Lead removal at trace concentrations from water by inactive yeast cells”, Communications Earth & Environment 3, 132, 1-9 (2022), the entire contents of which, together with the accompanying Supplementary Materials, is herein incorporated by reference.

In a particular implementation of this invention, inactive yeast biomass is used as a Pb biosorbent at the ppb scale. Biosorption of Pb and other heavy metals can be influenced by several factors, including yeast cell age and culture conditions, initial solution pH and solution pH at equilibrium, initial metal ion and initial biomass concentrations, and the presence of various ligands. For this purpose, the ideal conditions for yeast growth and drying were identified. The stoichiometry, equilibrium, and selectivity of the yeast biomass were measured and characterized under different pH values, trace initial Pb concentrations (at the range of 20-1,000 ppb), and biosorbent dosages.

A strain of the common yeast, Saccharomyces cerevisiae (S. cerevisiae), was selected as a preferred biosorbent for an implementation of the present invention, due to its unique nature compared to other microorganisms and fungal species [Wang, J. & Chen, C., “Biosorption of heavy metals by Saccharomyces cerevisiae: A review”, Biotechnol. Adv. 24, 427-451 (2006)]. S. cerevisiae is a nonpathogenic microorganism, which can be easily cultivated at large scales or obtained in large quantities as a waste or byproduct of various industrial settings. It can effectively remove Pb at ppm initial concentrations, and the fact that it has been fully studied as a model system in molecular biology facilitates the investigation of molecular mechanisms involved in biosorption.

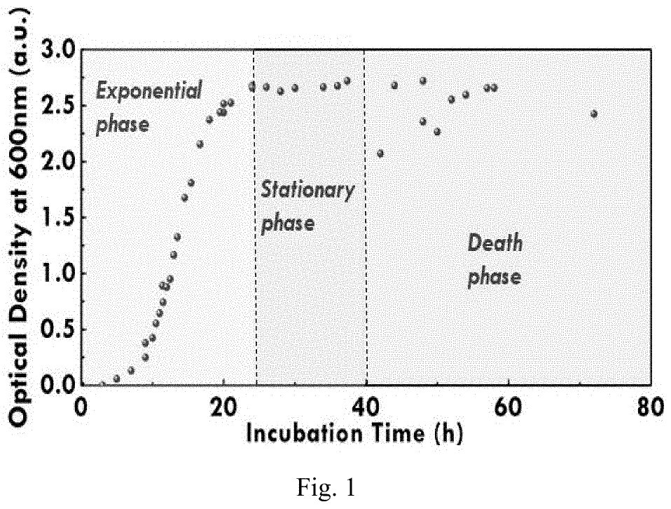

The Saccharomyces cerevisiae Meyen ex E. C. Hansen M Y A-796 strain used was purchased from the American Type Culture Collection (ATCC, Virginia, USA). This strain was selected because it is a biodegradable, inexpensive adsorbent that is widely used in various industrial settings, can be easily obtained, even as a by-product of fermentation industries, and can be cultivated at large scales [Sun, G. L., Reynolds, Erin. E. & Belcher, A. M.' “Using yeast to sustainably remediate and extract heavy metals from waste waters”, Nat. Sustain. 3, 303-311 (2020); Ojima, Y. et al., “Recovering metals from aqueous solutions by biosorption onto phosphorylated dry baker's yeast”, Sci. Rep. 9, 225 (2019); Wang, J. & Chen, C., “Biosorption of heavy metals by Saccharomyces cerevisiae: A review”, Biotechnol. Adv. 24, 427-451 (2006)]. The materials used, and the processes and steps performed to develop the yeast biomaterial and test it for Pb biosorption are briefly presented in FIGS. 1-4 and are described in detail in the following sections.

Growth and harvesting of yeast cells. YM agar and broth media (ATCC 200 YM Medium, ATCC, Virginia, USA) were used for the yeast cultures. To monitor the growth rate of the cells over time and identify their ideal harvesting time the yeast growth curve was obtained. Yeast cells were cultivated for 72 hours, incubated (Multitron Standard incubator shaker, INFORS HT, USA) at 30° C. and 200 rpm in 2 L Erlenmeyer flasks with shallow medium content (100-200 mL). 1 mL samples were taken from the culture at set time intervals to measure their optical density at 600 nm wavelength (OD600), using 2 ml non-frosted cuvettes (VWR Standard Spectrophotometer Cuvettes, VWR, USA) and a tabletop ultraviolet—visible spectrophotometer (NanoPhotometer NP80, Implen, Munich, Germany). FIG. 1 depicts a measured growth curve of the yeast, with absorbance accuracy being about 1.75% of reported values. Based on the developed growth curve, it was decided to harvest the cells at the first 18 hours of cultivation, i.e., at the peak of their exponential growth phase, for optimal biosorptive capacity. Yeast cells were harvested by centrifugation at 1 g/2000 rpm for 10 min (Allegra X15-R Centrifuge, USA).

Yeast biomass washing. Harvested cells underwent two washes with Type I ultrapure water (Sigma-Aldrich, Millipore Sigma, USA) to remove medium residues and metabolites before being lyophilized (freeze-dried), converted into powder, and used in biosorption experiments. For each wash, cells were centrifuged (1 g and 2,000 rpms for 10 mins, Beckman Coulter Allegra X15-R Centrifuge; rotor: SX4750A) and resuspended in ultrapure water. To identify the optimum number of washes, samples from the pure liquid culture medium, and the supernatants from the harvested liquid culture and from ultrapure water washed cells after centrifugation were analyzed using High-Performance Liquid Chromatography (HPLC—Aminex HPX-87H Column).

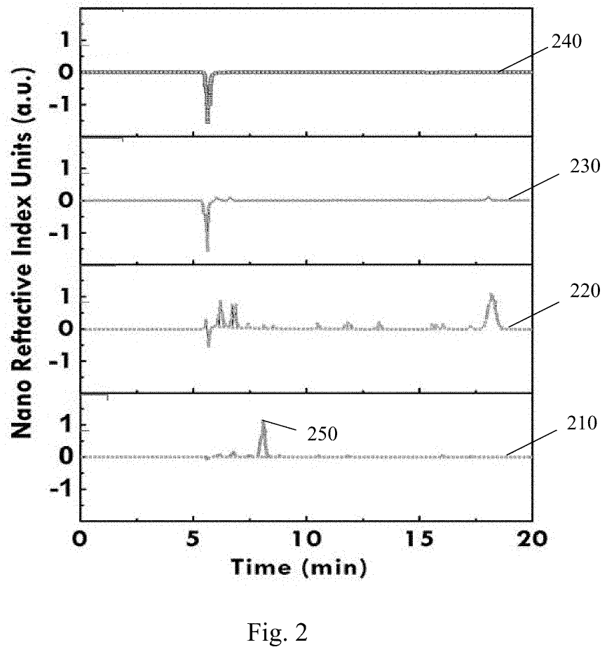

FIG. 2 is a graph of High-Performance Liquid Chromatography (HPLC) results, performed to identify the number of washes with ultrapure water required to remove medium residues and metabolites form harvested yeast cells before being used in biosorption experiments and indicating that after two washes with ultrapure water there is no presence of organic compounds in the analyzed solutions. Shown in FIG. 2 are curves for pure culture medium (pH ˜6.2) 210, supernatant of harvested liquid culture (pH ˜4.7) 220, supernatant after the first wash of yeast cells with ultrapure water (pH ˜5.3) 230, and supernatant after the second wash of yeast cells with ultrapure water (pH ˜5.8) 240.

As shown in FIG. 2, the pure culture medium had a peak 250 at 8 mins representing glucose, while in the supernatant of the harvested liquid culture, glucose was not present and ethanol was produced (peak at 18 mins). In the supernatant after the first wash of yeast cells with ultrapure water 230, there were almost no peaks representing the presence of sugars or organic acids in the solution, while in the supernatant after the second wash 240, there were no peaks at all. Therefore, it was decided to wash the harvested cells twice to ensure absence of organic compounds that might affect biosorption experiments.

Yeast biomass lyophilization. Inactive yeast cells were used, as they have been proven to have the same or even better uptake capacity of metal ions compared to active ones. Inactive cells can overcome several limitations which are difficult to control when active cells are used, including that maintenance of culture purity and solution toxicity is not a concern, there is no sensitivity to extreme pH values or higher metal ion concentrations, and biomass growth and viability does not need to be satisfied [Tsezos, M., “Biosorption: A Mechanistic Approach”, Geobiotechnology I. Metal-related Issues (eds. Schippers, A., Glombitza, F. & Sand, W.) 173-209, Springer-Verlag (2014); Wang, J. & Chen, C., “Biosorption of heavy metals by Saccharomyces cerevisiae: A review”, Biotechnol. Adv. 24, 427-451 (2006)].

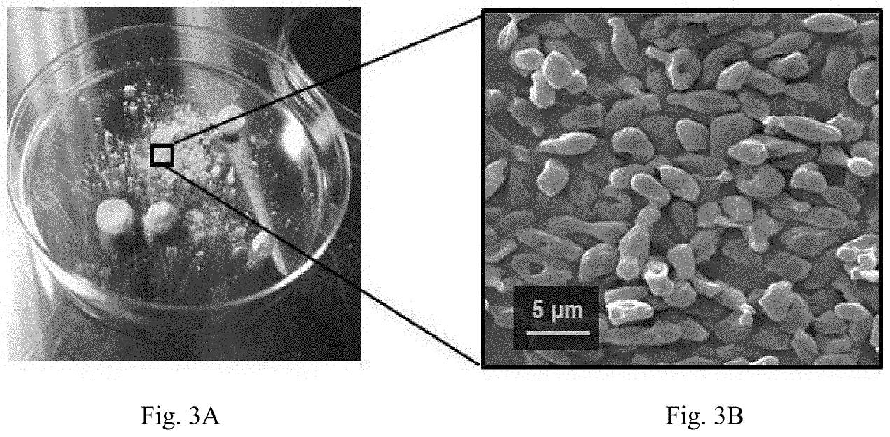

Lyophilization/freeze-drying is considered to be the best method to inactivate yeast cells and at the same time maintain their three-dimensional cell structure intact. Harvested, washed cells were kept at −30° C. for 24 hours and then inserted in the freeze dryer (Freezone 6 Liter Manifold, Labconco, USA). Lyophilization cycles (temperature: <−40° C., pressure: <0.371 mbar) lasted at least 50 hours. Lyophilized cells in powder form were stored in a desiccator containing silica gel at room temperature. Freeze-dried biomass was weighted using an analytical balance of 0.1 mg precision and resolution (AB104-S analytical balance, Mettler Toledo, USA). FIG. 3A is a photograph of yeast powder after lyophilization and FIG. 3B is SEM imaging of freeze-dried yeast cells (scale bar: 5 μm).

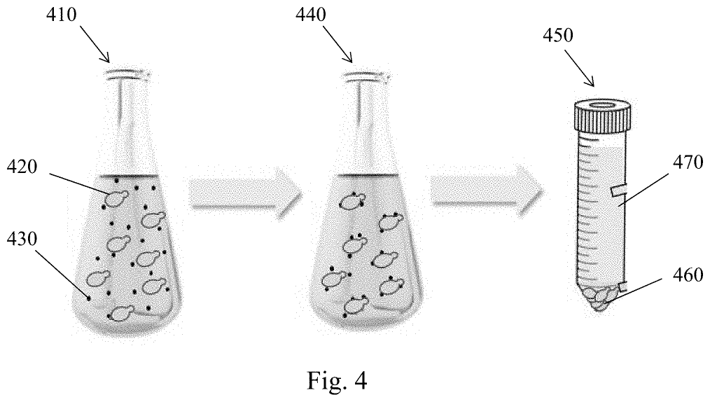

Batch biosorption experiments. Kinetic and equilibrium sorption batch contact experiments were conducted using 2L Erlenmeyer flasks. The yeast powder biomaterial was added in 200 mL of Type I ultrapure water spiked with lead(II) nitrate [Pb(NO₃)₂] (Sigma-Aldrich, Millipore Sigma, USA) to achieve initial Pb²⁺ concentrations of max 1,000 ug/L (ppb). FIG. 4 depicts the main steps of example kinetic and equilibrium experiments involving the addition 410 of freeze-dried yeast cells 420 in Pb 430-containing aqueous solutions, the adsorption 440 of Pb ions, and the separation 450 of biomass 460 and supernatant 470 after the required contact time via centrifugation for further analyses.

The initial solution pH was adjusted to the required values with 69% nitric acid (HNO₃) (ARISTAR PLUS, VWR Chemicals BDH, VWR, USA) and 0.1 M sodium hydroxide (NaOH) (Carolina Biological, USA). To accurately measure the pH of ultrapure water solutions, a glass-body electrode suitable for ion-weak samples was used (InLab Pure Pro-ISM, Mettler Toledo, USA). Flasks were incubated at 2009 rpms and 25° C. After the required contact time (determined by the kinetic experiments), solid and liquid phases were separated via centrifugation (10 mins at 2,000 rpm/1 g). Residual Pb²⁺ concentrations in the supernatant were measured using inductively coupled plasma—mass spectrometry (ICP-MS) (7900 ICP-MS system, Agilent, USA), following standard operating procedures (Pb²⁺ calibration standards and Bismuth internal standard, Agilent, USA). All experiments were carried out in triplicates and mean values are reported. For all experiments, a control sample of just ultrapure water spiked with Pb(NO₃)₂ was measured to act as a reference for the initial Pb²⁺ concentration, C₀, in the solution. Pb²⁺ removal measurements were calculated by taking the ICP-MS measurements of the supernatants and subtracting from this the reference to determine the quantity of metal adsorbed by yeast biomass. Type I ultrapure water alone was also tested via ICP-MS to make sure that there was no Pb²⁺ present in the aqueous matrix.

The amount of Pb²⁺ uptake from the biomatrial was calculated using Equation 1:

$${q}_{t} = ( \frac{{c}_{o} - {c}_{t}}{m} ) * V \qquad [ 1 ]$$

where qₜ is the Pb²⁺ mass (μg) adsorbed per g of yeast biomass after t contact time (μg/g); C₀ is the initial Pb²⁺ concentration in the aqueous solution (m/L); Cₜ is the residual Pb²⁺ concentration in the solution after t contact time (m/L); m is the dry weight of yeast biomass in the solution (g); and Vis volume of the aqueous solution (L). If equilibrium has been reached and Cₜ is Cₑ, then qₜ is qₑ. [Tsezos, M., “Biosorption: A Mechanistic Approach”, Geobiotechnology I: Metal-related Issues, 173-209, Springer-Verlag (2014); Wang, J. & Chen, C., “Biosorption of heavy metals by Saccharomyces cerevisiae: A review”, Biotechnol. Adv. 24, 427-451 (2006)].

In addition, advanced characterization techniques, i.e. scanning electron microscopy (SEM), transmission electron microscopy (TEM), X-ray photoelectron spectroscopy (XPS), and Fourier transform infrared spectroscopy with attenuated total reflectance (ATR-FTIR), were used to characterize the biomass and identify the functional groups and biomass sites responsible for Pb uptake. Each experiment was run in triplicate and mean values are reported, while control flasks without containing biomass were used in all experiments.

Effect of solution pH on Pb speciation and uptake. Solution pH is a key parameter influencing the biosorptive uptake capacity, as it affects the chemistry and speciation of both the potential metal-uptaking functional groups in the biomass, and of the hydrolyzed Pb ionic forms. Pb speciation in the solution is also affected by Pb concentration at any given pH and oxidation state [Tsezos, M., “Biosorption: A Mechanistic Approach”, Geobiotechnology I: Metal-related Issues (eds. Schippers, A., Glombitza, F. & Sand, W.) 173-209, Springer-Verlag (2014); Baes, C. F. & Mesmer, R. E., The hydrolysis of cations, Wiley (1976)]. To quantify the resulting Pb speciation after the hydrolysis of Pb(NO₃)₂ in a wide pH range (3-13), at 25° C., and at a given initial concentration, C₀, of 100 ppb, the ChemEQL v3.2 software was used [Eawag, “ChemEQL—a software for the calculation of chemical equilibria”, https://www.eawag.ch/en/department/surf/projects/chemeql (2021)].

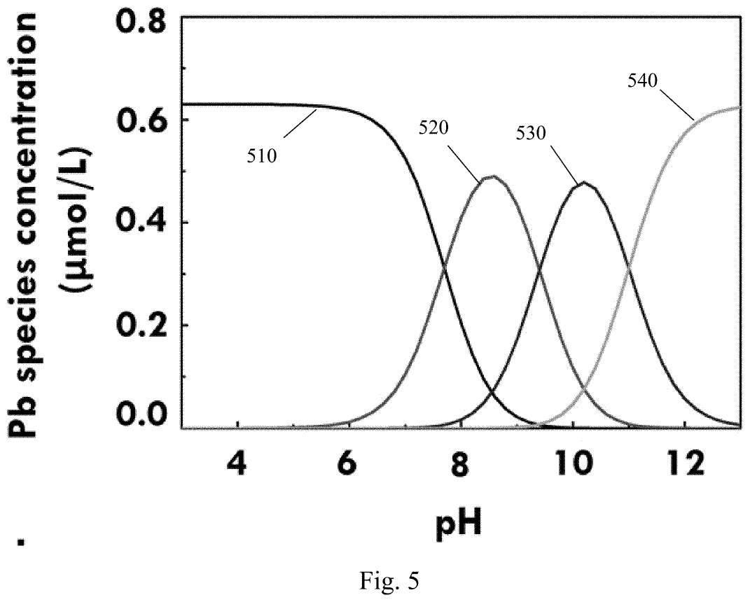

FIG. 5 is a graph of the distribution of Pb(NO₃)₂ hydrolysis products at 25° C. and 1 uM Pb(NO₃)₂. Shown in FIG. 5 are curves for Pb²⁺510, PbOH⁺520, Pb(OH)₂ 530, and Pb(OH)₃ 540. Shown in FIG. 5, Pb²⁺ is the dominant species until pH reaches 5.8, where lead hydroxides (e.g., PbOH⁺) are beginning to form

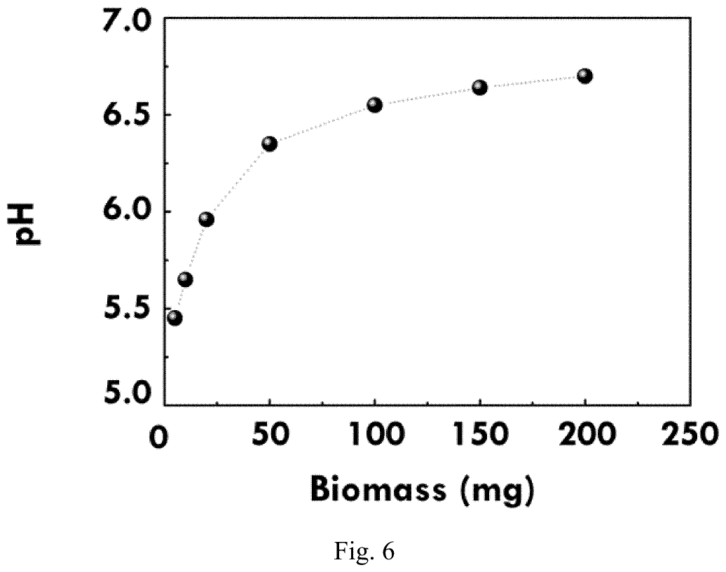

It was observed that solution pH increases with the addition of biomass and plateaus for biomass values greater than 0.10 g. FIG. 6 is a graph showing the increase in solution pH due to yeast biomass addition; error: ±0.06 pH units.

Therefore, to assess the effect of solution pH on the biomass uptake capacity (q), the initial pH of the aqueous solution (before biomass addition) was adjusted to pH values within the range of 3-7, and then 0.005 g of yeast biomass was added to moderate the anticipated pH increase due to biomass addition. Pb²⁺ concentrations and pH values were measured both before biomass addition and after Pb²⁺ (contact time of 24 h).The biomass Pb²⁺ uptake capacity greatly increased as the initial solution's pH was increased from 3 to 5.

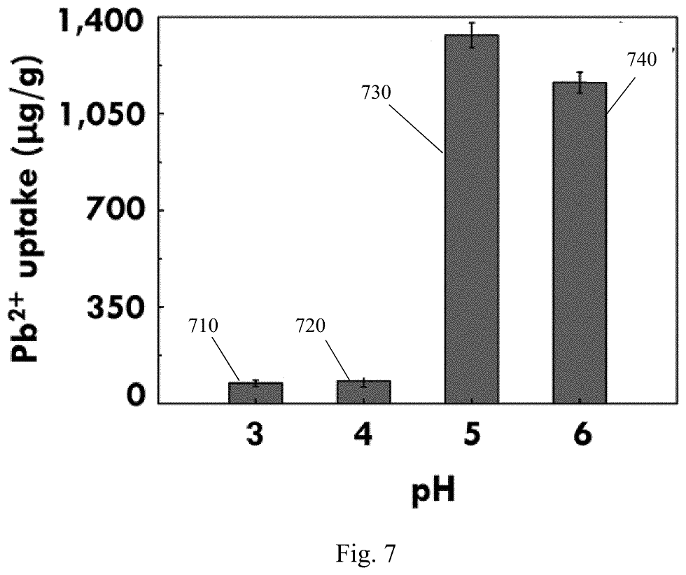

FIG. 7 is a graph depicting the effect of initial pH of solution on Pb²⁺ uptake for 0.005 g of yeast biomass, C₀: 100 ppb, and initial pH3 710, pH4 720, pH5 730, and pH6. The final pH of solutions after biosorption was pH3: 3.1, pH4: 4.1, pH5 5.5, and pH6 6.2; error: ±0.06 pH units. For pH ≥6.0 the measured initial Pb²⁺ concentration was notably lower than the known amount added to the solutions, indicating loss of soluble Pb analytes due to precipitation. This is validated by the formation of lead hydroxides after pH 5.8 (FIG. 5).

The increase of solution pH by biomass addition and of biosorption capacity with the increase in pH could be attributed to a potential protonation of the functional groups of yeast biomass at pH values below the pH point of zero charges, i.e., the pH at which the overall biomass surface charge is zero. At pH values below the pH point zero, the biomaterial may exhibit an overall positive charge, thus attracting negatively charged species and not adsorbing Pb²⁺ cations resulting in lower q values, while at higher pH values the biomass surface could acquire negative charges leading to increased Pb²⁺ uptake [Tsezos, M., “Biosorption: A Mechanistic Approach”, Geobiotechnology I: Metal-related Issues (eds. Schippers, A., Glombitza, F. & Sand, W.) 173-209, Springer-Verlag (2014)]. Indeed, the point of zero charges for S. cerevisiae cells is reported in the literature [Saitoh, N., Noruma, T. & Konishi, Y., “Sustainable use of precious and rare metals through biotechnological recycling”, REWAS 2019: Manufacturing the Circular Materials Economy (The minerals, metals & materials series), 107-114, Springer, (2019); Dengis, P. B., Nelissen, L. R. & Rouxhet, P. G., “Mechanisms of yeast flocculation: Comparison of top-and bottom-fermenting strains”, Appl. Environ. Microbiol. 61, 718-728 (1995)] to be around pH 4.0, hence, the surface of the yeast cells is possible to carry negative charges after this pH value, attracting Pb²⁺ cations. However, such an approach assumes a rather simplistic electrostatic attraction driving mechanism, which has been shown not to be the only case in biosorption [Tsezos, M., “Biosorption: A Mechanistic Approach”, Geobiotechnology I: Metal-related Issues (eds. Schippers, A., Glombitza, F. & Sand, W.) 173-209, Springer-Verlag (2014)].

Based on these results, pH 5 was proven to be the most suitable value for Pb biosorption, where soluble Pb²⁺ are the most dominant species in the solution and q is maximized. All the experiments described in the following sections were performed after adjusting the initial solution pH to 5. Solutions were then incubated at 25° C. and agitated at 200 rpm

The effects of pH thus observed can be reasonably expected to be generalizable to a number of similar organisms due to the highly similar, and in many cases identical, biophysical principles governing the rates of adsorption kinetics. The invention is therefore not limited to the specific embodiment reduced to practice, as it will be clear to one of skill in the art that many other microorganisms may be employed in the method of the invention including, but not limited to, Bacillus subtilis, Bacillus cereus, E. coli, Fischerella ambigua (algae), Trametes versicolor, Pleurotus ostreatus, Rhizopus arrhizus, and Schizosaccharomyces pombe.

Beyond the biophysical similarities noted above, it will be clear to one of skill in the art that only minor modifications, well-known to those of skill in the art, to the specific conditions utilized in the specific implementation and embodiments described herein will be needed in order to successfully employ organisms other than that used in the example embodiment in the method of the invention. It will further be clear to one of skill in the art that fewer modifications will be required for those organisms more closely resembling the exemplar, and that the successful use of other organisms in the invention may require additional modifications as the dissimilarities extend to critical conditions. For example, super-sensitivity to pH for an organism might necessitate pre-treatment to buffer the aqueous solution to within life-supporting pH ranges. Such modifications are well within the knowledge of one of ordinary skill in the art of the invention.

In cases where the performance of a selected organism is not optimized by the set of available pH, temperature, salinity and other physicochemical metrics of the solution to be treated, the organisms themselves may be subjected to directed adaptation via any of the methods known, such as, but not limited to, MACE, PACE, and the strategies described in Evolthon including, but not limited to, the “United States of E. coli” [U.S. patent application Ser. No. 16/133,600 (Mershin et al., Sep. 17, 2018).]

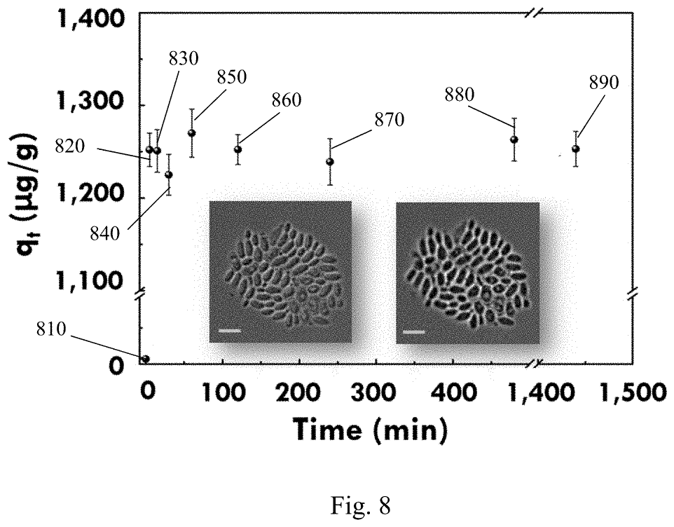

Adsorption kinetics and growth analysis of lyophilized yeast. Kinetic experiments were conducted to determine the change in Pb²⁺ concentration in the liquid phase as a function of contact time and identify the contact time required to attain equilibrium. Lyophilized yeast cells (m: 0.005 g) were added in 200 mL of aqueous solutions with initial Pb concentration, C₀, of 100 ppb and initial pH adjusted at 5. The flasks were shaken at 25° C. and 200 rpms for 24 hours and 2 mL samples were taken from each flask at specific time intervals: 0 mins, 5 mins, 15 mins, 30 mins, 1 h, 2 h, 4 h, 8 h, and 24 h. A control flask without biomass was also incubated under the same conditions to account for the atmospheric carbonate equilibria. Collected samples were analyzed using inductively coupled plasma-mass spectrometry (ICP-MS). It was observed that the biosorption process is a rapid process, as equilibrium was reached within the first five minutes of contact (FIG. 8).

In parallel the freeze-dried yeast cells were incubated for 24 hours under the exact same conditions followed during the kinetic experiment and observed under the optical microscope, while acquiring optical density (OD) measurements, in order to validate that the cells remain inactive during biosorption. Indeed, after 24 hours there was neither cell growth nor cell division observed.

FIG. 8 is a plot of the results from kinetic experiments at progressively long increments of contact time of 0 minutes 810, 5 minutes 820, 15 minutes 830, 30 minutes 840, 1 hour 850, 2 hours 860, 4 hours 870, 8 hours 880, and 24 hours 890 for 5 mg yeast biomass with C₀ of 100 ppb Pb²⁺, indicating the rapid biosorption process, with qₜ: Pb²⁺ uptake capacity of yeast biomass at different time intervals (μg of Pb²⁺ by g of biomass). Time course micrographs of lyophilized S. cerevisiae cells incubated in the same aqueous solution at 0 hrs and 24 hrs are shown, validating that there is no growth or division of cells during the experiments (scale bar: 8 μm).

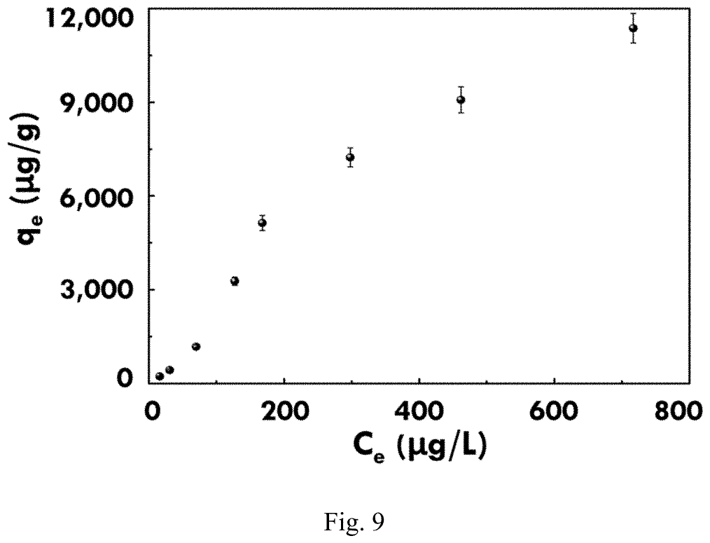

Adsorption isotherm. Aqueous solutions (0.2 L) with different initial Pb²⁺ concentrations (C₀: 20, 40, 100, 200, 300, 500 and 1,000 ppb), containing the same biomass dosage (m: 0.005 g) and with initial pH adjusted to 5 were shaken at 25° C. and 200 rpms for 1 hour. The equilibrium concentrations, Cₑ, were measured after the biosorption experiment and the biomass uptake capacity, qₑ, was calculated using Equation 1. FIG. 9 is a plot showing the adsorption isotherm at 25° C., following the Langmuir adsorption isotherm model; qₑ: Pb²⁺ uptake capacity of yeast biomass at equilibrium (μg of Pb²⁺ by g of biomass). The maximum qₑ measured was about 12 mg/g, for aqueous solutions with C₀ of 1000 ppb Pb²⁺.

The adsorption equilibrium isothermal model was developed, using the experimental Pb biosorption isotherm data. The data fit very well the Langmuir adsorption isotherm model (R²: 0.98), expressed by Equation 2:

$${q}_{e} = \frac{{q}_{m} * {K}_{L} * {C}_{e}}{1 + {K}_{L} * {C}_{e}} \qquad [ 2 ]$$

where K_{L}, (L/mg) is the ratio of the adsorption and desorption rates and qₘ (mg/g) is the maximum adsorption capacity estimated by the Langmuir model [Wang, J. & Guo, X., “Adsorption isotherm models: Classification, physical meaning, application and solving method”, Chemosphere 258, 127279 (2020)]. In the developed model, K_{L} and qₘ equals 1.5 L/mg 22.5 mg/g.

The Langmuir model [Volesky, B., “Advances in biosorption of metals: Selection of biomass types”, FEMS Microbiol. Rev., 14, 291-302 (1994); Wang, J. & Guo, X., “Adsorption isotherm models: Classification, physical meaning, application and solving method”, Chemosphere 258, 127279 (2020)] is one of the most widely used adsorption isotherm models, and is based on the following assumptions: (a) the biomass surface consists of adsorption sites, (b) metal ions interact only with the adsorption sites and not with each other, (c) adsorption is limited to a monolayer, and (iv) adsorption energy of all sites is identical and independent of the presence of metal ions on neighboring sites. However, in contrast to the ideal adsorption theory of gases, these models bear no mechanistic significance for biosorption, and this fit cannot provide any meaningful insight regarding the underlying mechanisms [ Tsezos, M., “Biosorption: A Mechanistic Approach”, Geobiotechnology I: Metal-related Issues, 173-209, Springer-Verlag, (2014); Wang, J. & Chen, C., “Biosorption of heavy metals by Saccharomyces cerevisiae: A review”, Biotechnol. Adv. 24, 427-451 (2006)].

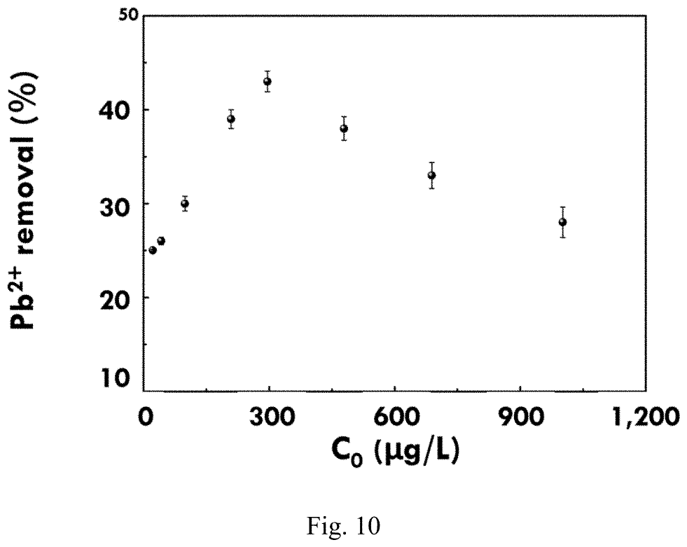

The Pb²⁺ percentage removal versus the initial Pb²⁺ concentration, C₀, is presented in FIG. 10. The Pb²⁺ removal for C₀ 20 ppb is around 25% and increases with the increase of C₀, reaching a maximum of 43% at C₀ 300 ppb. After this point, Pb²⁺ removal decreases gradually with the decrease of C₀, indicating that the optimum uptake capacity of the given yeast biomass quantity (0.005 g) is being reached around C₀ 300 ppb.

It will be clear to one of skill in the art that the methods and apparatus described here are also extendible to adsorption of organic and inorganic micropollutants beyond heavy metals, potentially benefiting from directed adaptation treatments to create adhesion and adsorption sites on cell walls ready to accept nanoparticles for instance. One method that is available for this is the M13 Bacteriophage Display Framework [Hess, Gaelen T., Cragnolini, Juan J., Popp, Maximilian W., Allan, Mark A., Dougan, Stephanie K., Spooner, Eric, Ploegh, Hidde T., Belcher, Angela M., and Guimaraes, Carla P., “M13 Bacteriophage Display Framework That Allows Sortase-Mediated Modification of Surface-Accessible Phage Proteins”, Bioconjugate Chem. 2012, 23, 7, 1478-1487 (July 3, 2012); Sun, G. L., Reynolds, E. E., and Belcher, A., M. Using yeast to sustainably remediate and extract heavy metals from waste waters. Nature Sustainability, (2020), https://doi.org/10.1038/s41893-020-0478-9]. In this instance, the selected organism would be transfected with genes allowing short (7-16 amino acid-long) peptide sequences to be expressed on the surface of (for instance) magnetotactic bacteria that are already attracted to metal but can also bind other micropollutants and sink to the bottom of a sludge pool.

SEM and TEM yeast biomass imaging. Extracellular and intracellular imaging of yeast cells was performed to observe potential changes in their structure after biosorption, using SEM and TEM imaging respectively. Yeast cells harvested from ultrapure water (C₀: 0 ppb Pb²⁺) served as control cells and were compared with yeast cells harvested from ultrapure water with C₀ 100 ppb.

For the SEM imaging yeast cells were freeze-dried again after biosorption. Samples of lyophilized yeast biomass (before and after Pb²⁺ biosorption) were coated in a 20 nm layer of gold (150TES Sputter Coater, EMS, USA), mounted onto carbon double-sided tape, and imaged with an SEM, using secondary electron detection at 10 kV beam voltage and varying magnifications (FlexSEM 1000, Hitachi, Tokyo, Japan).

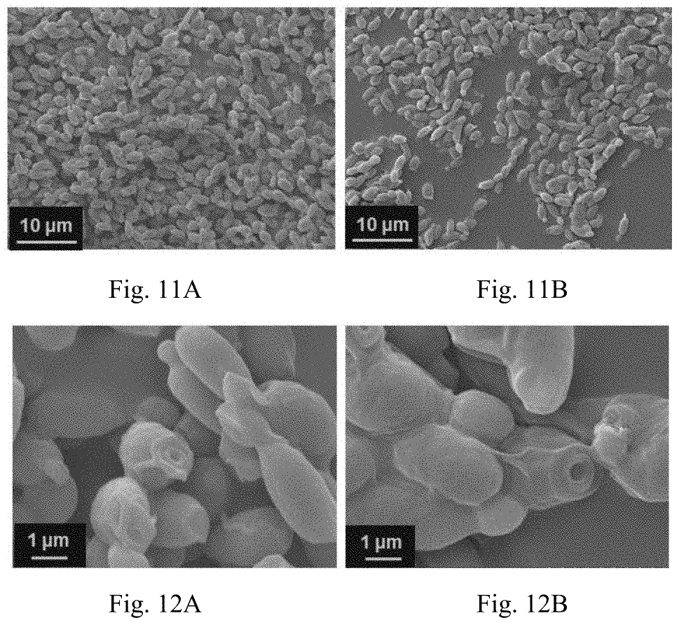

FIGS. 11A and 11B are SEM images depicting an overview of control yeast cells (C₀: 0 ppb; m: 0.005g; pH: 5) (FIG. 11A) and yeast cells after Pb²⁺ biosorption (C₀: 100 ppb; m: 0.005g; pH: 5) (FIG. 11B). FIGS. 12A and 12B are magnified SEM images showing individual control yeast cells (FIG. 12A) and individual yeast cells after Pb²⁺ biosorption (FIG. 12B). No morphological change was observed in the yeast cells after Pb²⁺ biosorption. The structure and dimensions of yeast cell wall and cytoplasm remained the same after Pb²⁺ biosorption. Yeast cell walls were measured using Image J software, taking the average measurements of 15 control and 15 Pb-exposed cells, and are about 180 nm, which is the typical cell wall thickness of the yeast strain used [Hudson, L. E. et al., “Characterization of the Probiotic Yeast Saccharomyces boulardii in the Healthy Mucosal Immune System”, PLOS ONE 11, e0153351 (2016)].

For the TEM imaging, harvested yeast cells underwent fixation, dehydration, resin embedding and sectioning. Harvested yeast cells were fixed with 2% glutaraldehyde and 2.5% paraformaldehyde in 100 mM sodium cacodylate buffer (EMS, USA) for 1 h at 4° C. Then cells were washed twice with 100 mM sodium cacodylate buffer, each time re-suspending them by vortexing, storing them at 4° C. for 15 mins, and centrifuging them for 10 mins at 1 g/2000 rpm. Then cells were resuspended in 100 mM sodium cacodylate buffer and stayed overnight at 4° C. Pelleted cells were post-fixed for 30 min at 4° C. with 1% osmium tetroxide in 0.1 M imidazole of pH 7.5 (EMS, USA). Osmium post-fixation was followed by washing the cells three times with 100 mM sodium cacodylate buffer, storing them each time for 15 mins at room temperature. The cells were then washed three times with 0.05 M maleate buffer of pH 5.15 (EMS, USA), storing them each time for 15 mins at room temperature. The cells were then stained in 2% uranyl acetate (EMS, USA) overnight, in the dark at room temperature. Stained cells were washed three times with 0.05 M maleate buffer of pH 5.15, storing them each time for 15 mins at room temperature. Then, the cells were serially dehydrated using 30%, 35%, 50%, 70%, 90%, 95%, and 100% ethanol solutions. Dehydrated cells were embedded in EMBED-812 resin (EMS, USA). Sections were cut using an ultramicrotome (EMUC7, Leica, Wetzlar, Germany) and a Diatome diamond knife at a thickness setting of 50 nm. The sections were examined using a TEM (Tecnai G2 Spirit TWIN, FEI, USA) at 120 kV.

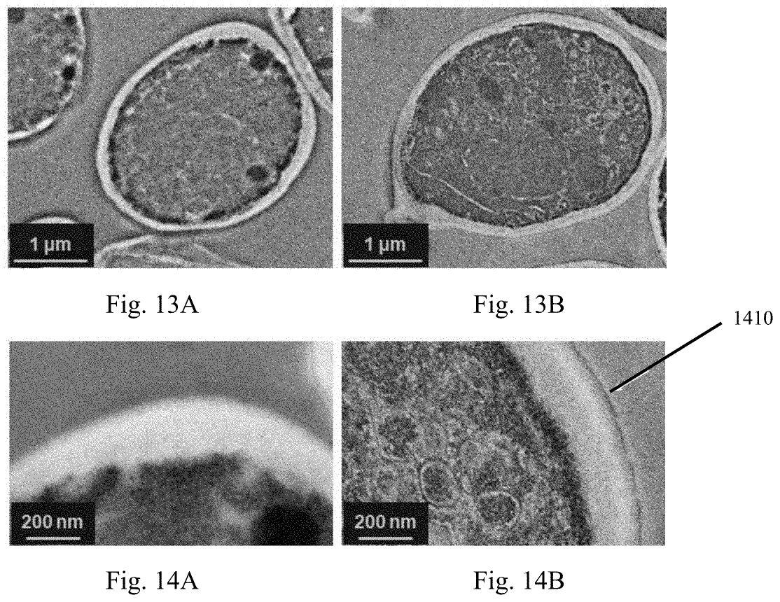

FIGS. 13A and 13B are TEM images of individual control yeast cells (C₀: 0 ppb; m: 0.005g; pH: 5) (FIG. 13A) and individual yeast cells after Pb²⁺ biosorption (C₀: 0 ppb; m: 0.005g; pH: 5) (FIG. 13B). FIGS. 14A and 14B are magnified TEM images of control yeast cell walls (FIG. 14A) and yeast cell walls after Pb²⁺ biosorption (FIG. 14B). In FIG. 14B, outer part of the cell wall 1410 has electron dense Pb-binding areas. As shown in FIGS. 13A-B and 14A-B, yeast cell walls became more electron dense after Pb²⁺ biosorption, indicating the binding of Pb²⁺ ions on them. In particular, a thin layer of Pb²⁺ can be observed on the outer part of the cell wall after biosorption.

Yeast biomass spectroscopy—ATR-FTIR and XPS yeast biomass characterization. Metal biosorption is reported to occur through interactions with functional groups native to the biomass cell wall [Goksungur, Y., “Biosorption of cadmium and lead ions by ethanol treated waste baker's yeast biomass”, Bioresour. Technol. 96, 103-109 (2005)]. ATR-FTIR technique was performed to identify functional groups present in the yeast biomass and detect changes in them after biosorption, indicating their involvement in Pb²⁺ adsorption. Freeze-dried control (C₀: 0 ppb) and Pb²⁺-exposed yeast cells (C₀: 100 and 1,000 ppb) were analyzed using a Thermo Fisher Scientific NicoletTM iS50 FTIR Spectrometer within the range of 400-4,000 cm ¹.

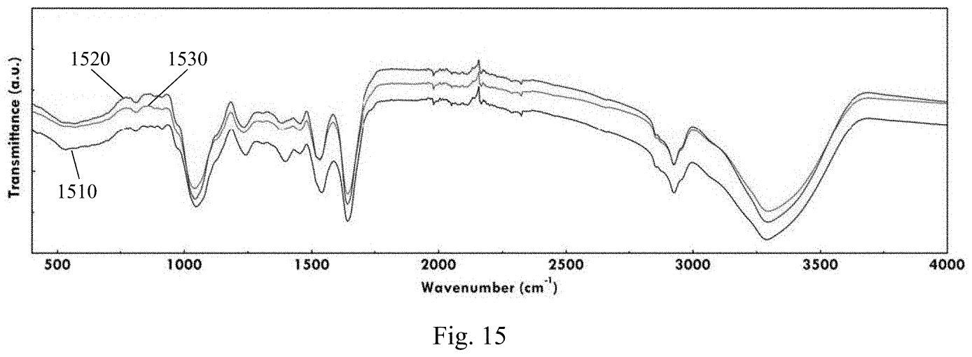

FIG. 15 is a graph depicting the full ATR-FTIR spectrum of yeast cells. Shown in FIG. 15 are plots for control yeast cells 1510 that have not been exposed to Pb²⁺(C₀: 0 ppb Pb²⁺), yeast cells after Pb2+ biosorption (C₀: 100 ppb) 1520, and yeast cells after Pb²⁺ biosorption (C₀: 1000 ppb) 1530. As shown in FIG. 15 changes were observed after biosorption in peaks representing C≡N and C≡C stretches, while peak shifts were detected corresponding to N—H in-plane bending from secondary protein amides, which overlaps with the C—N and NO₂ asymmetric stretching, to vibrational changes of the C—N amide group, and to C—O stretching in the esters and carboxylic acid groups. These changes indicate the contribution of amide and carboxylic acid groups to Pb²⁺ biosorption and the potential role of N in the yeast cell wall in Pb²⁺ binding. Detailed description of the ATR-FTIR results is provided in the following sections.

In all three samples, a very strong and broad hydroxyl peak is present near 3,300 cm⁻¹, representing the symmetric stretching vibration of the O—H bonds, while a sharp peak close to 1650 cm⁻¹ is attributed to carbonyl groups of proteins (C═O). The presence of both the hydroxyl (O—H) and the carbonyl (C═O) peaks indicates that the yeast biomass contains the carboxylic acid functionality (COOH), participating in hydrogen bonding. Absorption bands at 2,925 cm⁻¹ may result from the stretching vibrations of C—H (alkanes), as they are indicative of sp³ hybridized carbons bonded to hydrogens (Csp³-H) [Smith, B. C., Infrared Spectral Interpretation: A Systematic Approach, CRC Press (1999); De Rossi, A. et al., “Chromium (VI) biosorption by Saccharomyces cerevisiae subjected to chemical and thermal treatments”, Environ. Sci. Pollut. Res. 25, 19179-19186 (2018)].

Peaks at 2,247 cm⁻¹ and 2,173 cm⁻¹, which are present at the control yeast biomass, undergo a significant signal reduction after Pb biosorption, while new peaks appear after biosorption at 2,215 cm⁻¹,2,139 cm⁻¹ and 2,088 cm⁻¹. In addition, the peak at 2,043 cm⁻¹ becomes stronger after Pb biosorption, while the peak at 2,036 cm⁻¹ becomes weaker after biosorption. All these peaks represent C≡N and C≡C stretches, indicating that these bonds contribute to the biosorption process. Furthermore, a blue peak shift was observed after biosorption from 1,538 cm⁻¹ (control) to 1,530 cm⁻¹, corresponding to N—H in-plane bending from secondary protein amides, which overlaps with the C—N and NO₂ asymmetric stretching. In addition, a blue peak from 1,397 cm⁻¹ (control) to 1,383 cm⁻¹ was noticed, corresponding to vibrational changes of the C-N amide group, or of the N—O functional group. Furthermore, a blue peak shift from 1,241 cm⁻¹ to 1,234 cm⁻¹ corresponds to C—O stretching in the esters and carboxylic acid groups. These changes, which are presented in Table 1, indicate the contribution of amide and carboxylic acid groups on Pb biosorption, and the potential role of N in the yeast cell wall on Pb binding [Smith, B. C., Infrared Spectral Interpretation: A Systematic Approach, CRC Press (1999); De Rossi, A. et al., “Chromium (VI) biosorption by Saccharomyces cerevisiae subjected to chemical and thermal treatments”, Environ. Sci. Pollut. Res. 25, 19179-19186 (2018)].

TABLE 1

| Wavenumber | | | | Vibration/functional | |
|---|---|---|---|---|---|
| Control | | 100 ppb | 1,000 ppb | groups | Comments |
| 2,247 | cm⁻¹ | — | — | C≡N stretch | Peak disappeared after |
| | | | | | biosorption |
| | — | 2,215 cm⁻¹ | 2,215 cm⁻¹ | C≡C stretch | New peak appeared after |
| | | | | | biosorption |
| 2,173 | cm⁻¹ | — | — | C≡N, C≡C stretch | Peak disappeared after |
| | | | | | biosorption |
| | — | 2,139 cm⁻¹ | 2,139 cm⁻¹ | C≡C stretch | New peak appeared after |
| | | | | | biosorption |
| | — | 2,088 cm⁻¹ | 2,088 cm⁻¹ | C≡N, C≡C stretch | New peak appeared after |
| | | | | | biosorption |
| 2,043 | cm⁻¹ | 2,043 cm⁻¹ | 2,043 cm⁻¹ | C≡C, C≡C stretch | Peak became stronger after |
| | | | | | biosorption |
| 2,036 | cm⁻¹ | 2,036 cm⁻¹ | 2,036 cm⁻¹ | C≡N, C≡C stretch | Peak became weaker after |
| | | | | | biosorption |
| 1,962 | cm⁻¹ | — | — | C—H overtone, weak | Peak disappeared after |
| | | | | (aromatic compound) | biosorption |
| 1,741 | cm⁻¹ | — | — | C═O stretch (aliphatic) | Peak disappeared after |
| | | | | from esters group | biosorption |
| 1,538 | cm⁻¹ | 1,531 cm⁻¹ | 1,530 cm⁻¹ | N—H in plane bend; | Peak shifted after |
| | | | | NO₂ asymmetric | biosorption |
| | | | | stretch | |
| 1,397 | cm⁻¹ | 1,383 cm⁻¹ | 1,384 cm⁻¹ | C—H bend; C—N | Peak shifted after |
| | | | | stretch; NO₂ | biosorption |
| | | | | symmetric stretch; | |
| | | | | N—O stretch | |
| 1,241 | cm⁻¹ | 1,234 cm⁻¹ | 1,234 cm⁻¹ | C—N stretch (aliphatic | Peak shifted after |
| | | | | amines) | biosorption |
| 854 | cm⁻¹ | — | — | C═C bending | Peak disappeared after |
| | | | | | biosorption |
| 532 | cm⁻¹ | — | — | PO4-3 bend | Peak disappeared after |
| | | | | | biosorption |

The presence of both the hydroxyl (O—H) and the carbonyl (C═O) peaks indicates that the yeast biomass contains the carboxylic acid functionality (COOH), participating in hydrogen bonding. Absorption bands at 2,925 cm⁻¹ may result from the stretching vibrations of C—H (alkanes), as they are indicative of sp^{a} hybridized carbons bonded to hydrogens (Csp³-H) [Smith, B. C., “Infrared Spectral Interpretation: A Systematic Approach”, CRC Press (1999); De Rossi, A. et al., “Chromium (VI) biosorption by Saccharomyces cerevisiae subjected to chemical and subjected to chemical and thermal treatments”, Environ. Sci. Pollut. Res. 25, 19179-19186 (2018)], establishing the relationship between physicochemical parameters and yeast species via an unmutable physical principle governing hydrogen bonding, a critical physical pathway for this mechanism.

The chemical composition of the yeast surface before (C₀: 0 ppb) and after Pb²⁺ biosorption (C₀: 100 and 1000 ppb) was also analyzed by X-ray photoelectron spectroscopy (XPS) to further explore potential changes on the functional groups of the yeast cell walls that can indicate their contribution to Pb²⁺ uptake. XPS can provide an elemental and functional analysis of the surface of solids over a thickness of 2-5 nm [Dengis, P. B., Genet, M. J. & Rouxhet, P. G., “Microbial Cells by XPS: Analysis of Brewing Yeast Saccharomyces cerevisiae”, Surf Sci. Spectra 4, 21-27 (1996); Dengis, P. B. & Rouxhet, P. G., “Preparation of yeast cells for surface analysis by XPS”, J. Microbiol. Methods 26, 171-183 (1996)]. Freeze-dried control (C₀: 0 ppb) and Pb-exposed yeast cells (C₀: 100 and 1000 ppb) were analyzed with a Thermo Scientific K-Alpha XPS. Freeze-dried biomass was taken with a spatula and loaded on a stainless steel XPS stage (5 mm width). All three samples were analyzed in the same XPS run (spot size 400 pm). The data processing and peak fitting was done using the Thermo Avantage software and the Powel fitting algorithm.

The yeast surface is mainly composed of carbon (C), oxygen (O), and nitrogen (N) [Dengis, P. B., Genet, M. J. & Rouxhet, P. G., “Microbial Cells by XPS: Analysis of Brewing Yeast Saccharomyces cerevisiae”, Surf Sci. Spectra 4, 21-27 (1996)]. Therefore, C 1 s, O 1 s, N 1 s core levels spectra were recorded together with the Pb 4f. Table 2 presents surface atomic concentrations of yeast cells before and after Pb²⁺ biosorption, as measured during the XPS analysis.

TABLE 2

| | Atomic concentration (%) | | |
|---|---|---|---|
| | Control | Cells after biosorption | Cells after biosorption |
| Element | cells | (C₀: 100 ppb Pb²⁺) | (C₀: 1000 ppb Pb²⁺) |
| Carbon | 70.9% | 71.6% | 62.2% |
| Oxygen | 27.8% | 26.9% | 36.2% |
| Nitrogen | 1.3% | 1.5% | 1.6% |

Significant changes among the control and the Pb²⁺-exposed yeast were only observed for the C 1 s spectrum, while changes in the Pb spectra could not be detected as the analyzed trace Pb²⁺ concentrations were below the detection limit of the instrument. Table 3 presents XPS peaks profile data for yeast cells before and after Pb²⁺ biosorption.

TABLE 3

| | Binding energy | Full width at half | Area (P) |
|---|---|---|---|
| Peak | (eV) | maximum - FWHM (eV) | CPS · eV |
| | Control cells | | |
| C—C | 284.3 | 1.5 | 142,838 |
| C—N | 285.9 | 1.3 | 98,326 |
| O—C═O | 287.5 | 1.5 | 36,566 |
| C═O | 288.6 | 0.9 | 3,442 |
| | Cells after biosorption (C₀: 100 ppb Pb²⁺) | | |
| C—C | 284.3 | 1.9 | 72,014 |
| C—N | 285.9 | 1.1 | 67,571 |
| O—C═O | 287.5 | 2.1 | 68,559 |
| C═O | 288.6 | 1.5 | 3,204 |
| | Cells after biosorption (C₀: 1,000 ppb Pb²⁺) | | |
| C—C | 284.3 | 2.1 | 17,129 |
| C—N | 285.9 | 1.2 | 7,227 |
| O—C═O | 287.5 | 2.2 | 9,679 |
| C═O | 288.6 | 1.4 | 627 |

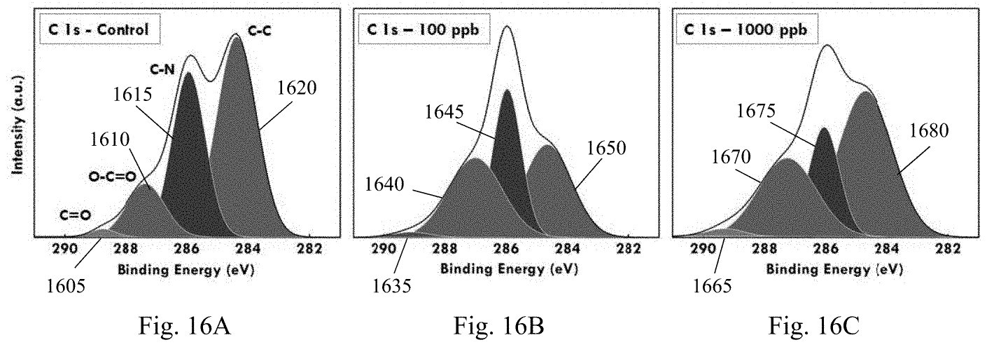

Deconvolution of the C is spectrum into Gaussian-shaped lines was performed to identify possible chemical bonds between C, O and N (FIGS. 16A-C). FIGS. 16 A-C are graphs depicting the results of XPS analysis of yeast cells. FIG. 16A depicts results for control cells that have not been exposed to Pb, FIG. 16B depicts results for yeast cells after Pb²⁺ biosorption with C₀ of 100 ppb Pb²⁺, and FIG. 16C depicts results for yeast cells after Pb²⁺ biosorption with C₀ of 1,000 ppb Pb²⁺. In FIGS. 16A-C, respective XPS peak assignments are C═O 1605,1635, 1665; O—C═O 1610, 1640, 1670; C—N 1615, 1645, 1675; and C—C 1620, 1650, 1680.

In all three samples the C 1 s peaks are decomposed to peaks at 284.3 eV, 285.9 eV, 287.5 eV and 288.6 eV, representing C—C (sp³ C), C—N, O—C═O, and C═O respectively. Survey scans used a pass energy of 50 eV while the core scans used a pass energy of 1.5 keV and were energy-calibrated using the C—C bond energy at 284.3 eV. The magnitude and shape of all observed bonds has radically changing after Pb biosorption, particularly for C—C, C—N, and O—C═O bonds. These changes indicate the contribution of carboxylic acid and amide groups to Pb²⁺ adsorption, which is consistent with the ATR-FTIR results. Changes in carboxyl, amino, hydroxyl and amide groups of protein and carbohydrate fractions of yeast cell walls have been also reported in the literature for higher initial Pb²⁺ concentrations (ppm scale) [Wang, J. & Chen, C., “Biosorption of heavy metals by Saccharomyces cerevisiae: A review”, Biotechnol. Adv. 24, 427-451 (2006); Machado, M. D., Janssens, S., Soares, H. M. V. M. & Soares, “E. V. Removal of heavy metals using a brewer's yeast strain of Saccharomyces cerevisiae: advantages of using dead biomass”, J. Appl. Microbiol. 106, 1792-1804 (2009)].

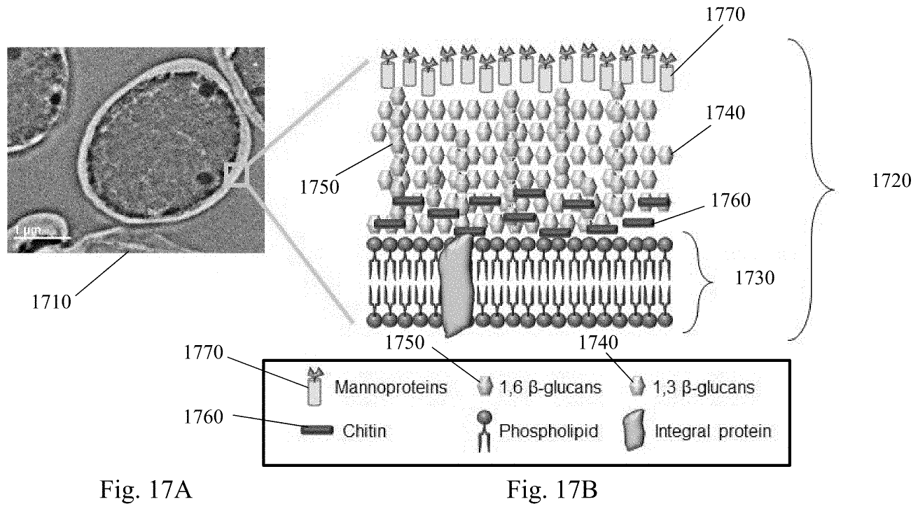

Contribution of chitin to Pb²⁺ adsorption. The above analyses indicate that the cell wall of S. cerevisiae plays a vital role in Pb²⁺ biosorption. FIGS. 17A and 17B are a TEM image 1710 of a Saccharomyces cerevisiae cell (FIG. 17A) and a schematic of Saccharomyces cerevisiae cell wall structure (FIG. 17B), respectively. The yeast cell wall 1720 has a complex macromolecular structure with a layered organization within cell wall membrane 1730, including an amorphous inner and a fibrillar outer layer [Machado, M. D., Janssens, S., Soares, H. M. V. M. & Soares, “E. V. Removal of heavy metals using a brewer's yeast strain of Saccharomyces cerevisiae: advantages of using dead biomass”, J. Appl. Microbiol. 106, 1792-1804 (2009)]. The inner layer mainly consists of β-glucans 1740, 1750 and chitin 1760. The outer layer consists predominantly of mannan polymers, highly glycosylated and linked to proteins (mannoproteins 1770) [Orlean, P., “Architecture and Biosynthesis of the Saccharomyces cerevisiae Cell Wall”, Genetics 192, 775-818 (2012)].

Several sources suggest that the chitin amine nitrogen is responsible for heavy metals sequestering at the ppm scale [Tsezos, M., “Biosorption: A Mechanistic Approach”, Geobiotechnology I: Metal-related Issue, 173-209, Springer-Verlag, 2014; Tsezos, M. & Mattar, S. “A further insight into the mechanism of biosorption of metals, by examining chitin epr spectra”, Talanta 33, 225-232 (1986); Tsezos, M. & Volesky, B., “Biosorption of uranium and thorium”, Biotechnol. Bioeng. 23, 583-604 (1981)]. To further investigate this, the Pb²⁺ uptake capacity of chitin from shrimp shells, which has similar characteristics to the chitin found in the yeast cell wall [Di Mario, F., Rapana, P., Tomati, U. & Galli, E., “Chitin and chitosan from Basidiomycetes”, Int. J. Biol. Macromol. 43, 8-12 (2008); Afroz, M., Kashem, M., Hasan, N., Piash, K. M. & Islam, N., “Saccharomyces cerevisiae as an untapped source of fungal chitosan for antimicrobial action”, Appl. Biochem. Biotechnol. 193, 3765-3786 (2021); Acosta, N., Jiménez, C., Borau, V. & Heras, A., “Extraction and characterization of chitin from crustaceans”, Biomass Bioenergy 5, 145-153 (1993)], was assessed. 20 times more chitin than the maximum equivalent amount present in the 5 mg of yeast was used, i.e., 1.8 mg, considering a 30% dry weight of yeast cell wall and a 6% contribution of chitin per mass to it [Machado, M. D., Janssens, S., Soares, H. M. V. M. & Soares, “E. V. Removal of heavy metals using a brewer's yeast strain of Saccharomyces cerevisiae: advantages of using dead biomass”, J. Appl. Microbiol. 106, 1792-1804 (2009)]. This amount was added to an aqueous solution of 0.2 L with C₀ of 1000 ppb Pb²⁺ for 24 h at 200 rpm and 25° C. The same experiments were also run with 5 mg of yeast biomass and with 5 mg of chitin.

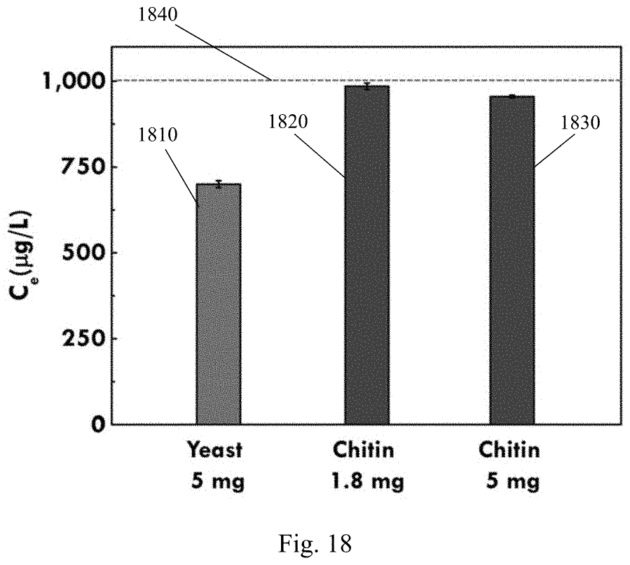

FIG. 18 is a graph illustrating chitin's contribution to Pb²⁺ adsorption, showing Pb²⁺ Cₑ after biosorption experiments with 5 mg of yeast biomass 1810, 1.8 mg chitin 1820, and 5 mg of chitin 1830. Dotted line 1840 represents C₀: 1000 ppb Pb²⁺. It was shown that chitin's Pb²⁺ uptake is negligible, as C₀ was reduced by less than 0.3% when the 1.8 mg of chitin was added, which is within the measurement error. Even when the chitin amount added was equal to the total yeast mass (5 mg), C₀ was only reduced by 3%, compared to the ˜30% reduction achieved by the yeast biomass. Hence, it can be concluded that chitin alone is not contributing to the Pb²⁺ biosorption process.

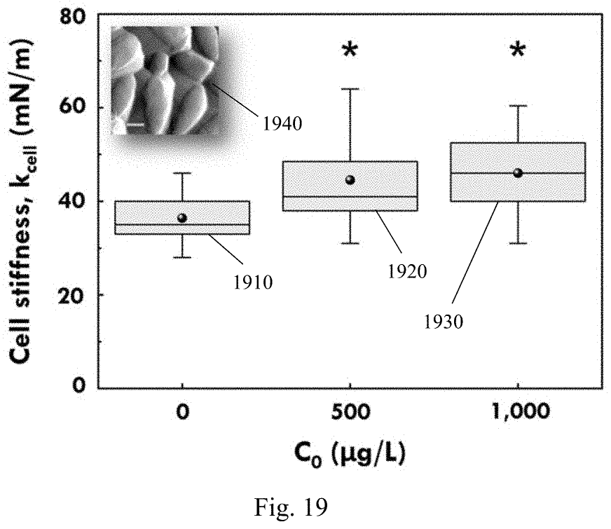

Yeast biomass nanomechanical characterization. Nanomechanical characterization was employed to investigate biosorption. The stiffness of the yeast cells was assessed before and after Pb²⁺ exposure, by conducting shallow (<10% of hydrated cell wall thickness) atomic force microscopy (AFM) indentations. FIG. 19 is a graph depicting results from AFM analysis of yeast cell stiffness. Fig.19 shows box and whisker plots of the cellular spring constant (k_{cell}) calculated from force-extension curves acquired from yeast cells incubated with C₀ of 0 ppb 1910, 500 ppb 1920, and 1000 ppb 1930 Pb²⁺. The boxed region indicates upper and lower quartiles for each data set, median is indicated by the horizontal line within the box, mean is indicated by the bullet point, and whiskers extend to high and low data points (n >70 force measurements from ≥8 cells per condition). Asterisks indicate p <le-5 by single factor ANOVA test between cell groups incubated with C₀ of 0 ppb, 500 ppb, or 1000 ppb Pb²⁺. AFM deflection retrace image of yeast cells is shown in inset 1940 (scale bar: 2 μm). Single-cell AFM mechanical testing showed a notable increase in the stiffness of cells following Pb²⁺ uptake.

As shallow AFM indentations provide an isolated cell wall response rather than a bulk cell response, AFM results confirm Pb²⁺ ion reinforcement of the yeast cell wall. It is possible that adsorption of a thin layer of Pb ²⁺ can act as a film on the cell surface that fuses the fibrillar structures together, which then effectively resists deformation more than the untreated cell wall. However, when yeast biomass is treated with solutions containing higher Pb²⁺ levels, mechanical stiffness is not noticeably increased (FIG. 19, C₀ of 500 vs 1000 ppb Pb²⁺). This cannot be attributed to a potential saturation of the yeast cell wall binding sites, as Pb²⁺ uptake increases substantially with the increase of C₀ from 500 to 1000 ppb Pb²⁺, not reaching saturation (FIG. 9). On the other hand, maximum Pb²⁺ removal is observed for C₀ of 300 ppb, and the Pb²⁺ uptake rate drops with increasing C₀ after this point, indicating lower adsorption efficiency (FIG. 10). While the exact mechanism for this stiffness change is yet to be determined, from the AFM experiments it can be concluded that the rate of yeast cell wall stiffening follows a similar pattern to that of the Pb²⁺ uptake rate with increasing C₀ (lower rates for C₀>300 ppb)

The above analysis of the invention demonstrates the biosorption isotherm and kinetics of initial Pb²⁺ concentration at the ppb scale, using lyophilized S. cerevisiae yeast cells as biosorbents. By comparing the results with prior studies of similar systems at the ppm scale, it can be concluded that the biosorption processes at the ppb scale happen faster. The fastest equilibrium attainment reported at the ppm scale is 10 min [Wang, J. & Chen, C., “Biosorption of heavy metals by Saccharomyces cerevisiae: A review”, Biotechnol. Adv. 24, 427-451 (2006)], while the present results show that equilibrium is achieved within the first 5 minutes of contact. The adsorption isotherm reported in FIG. 9 follows the same pattern as the adsorption isotherms reported in the ppm literature. Interestingly, the maximum Pb²⁺ uptake capacity of 12 mg/g reported herein is in the same range as the uptake capacities reported in the ppm literature for untreated inactive S. cerevisiae yeast, i.e., 2-30 mg/g, proving the suitability of this biomaterial as a biosorbent at the ppb scale. pH greatly affects the biosorption process in both scales.

The rapid biosorption and high Pb²⁺ uptake are advantageous for the large-scale application of this inexpensive and abundant biomaterial for the removal of trace heavy metals from water. The invention may also be applied to other heavy metal cations as well, such as copper (Cu²⁺) and cadmium (Cd²⁺).

From the performed analyses, it can be concluded that a one-step Pb²⁺ uptake process occurs, mainly due to the cell wall of S. cerevisiae, and in particular its carboxylic acid and amide groups. The cell wall of S. cerevisiae, and particularly its amino, carboxyl, and hydroxyl groups has been shown to greatly contribute to the biosorption of heavy metal cations at the ppm scale as well [Wang, J. & Chen, C., “Biosorption of heavy metals by Saccharomyces cerevisiae: A review”, Biotechnol. Adv. 24,427-451 (2006); Brady, D., Stoll, A. D., Starke, L. & Duncan, J. R., “Chemical and Enzymatic Extraction of Heavy Metal Binding Polymers from Isolated Cell Walls of Saccharomyces cerevisiae”, Biotechnol. Bioeng. 44,297-302 (1994)]. Although further elucidation of the exact uptake processes is needed, there is no indication of a multi-step uptake process, which is often reported in the ppm literature for non-living biomass, involving diffusion and/or accumulation of Pb²⁺ ions inside the cytoplasm [Wang, J. & Chen, C., “Biosorption of heavy metals by Saccharomyces cerevisiae: A review”, Biotechnol. Adv. 24,427-451 (2006); El-Sayed, M. T., “Removal of lead(II) by Saccharomyces cerevisiae”, AUMC 3875, Ann. Microbiol. 63,1459-1470 (2013); Suh, J. H., Yun, J. W. & Kim, D. S., “Comparison of Pb²⁺ accumulation characteristics between live and dead cells of Saccharomyces cerevisiae and Aureobasidium pullulans”, Biotechnol. Lett. 20,247-251 (1998)]. As the yeast cells are inactive, additional steps would require disruption of the yeast cell membrane, or changes in its permeability, allowing the penetration of Pb²⁺ ions. However, freeze-drying of yeast cells protects their structure and is not associated with loss of membrane integrity, in contrast with autoclaving [El-Sayed, M. T., “Removal of lead(II) by Saccharomyces cerevisiae”, AUMC 3875, Ann. Microbiol. 63,1459-1470 (2013); Suh, J. H., Yun, J. W. & Kim, D. S., “Comparison of Pb²⁺ accumulation characteristics between live and dead cells of Saccharomyces cerevisiae and Aureobasidium pullulans”, Biotechnol. Lett. 20, 247-251 (1998)], hence, the occurrence of additional uptake steps is unlikely.

The chitin experiment provides direct evidence of the importance of proteins and protein-carbohydrates of the outer and middle layers of the cell wall over the chitinous inner layers in Pb²⁺ biosorption. By excluding chitin as a biosorbent, mannoproteins and β-glucans are the potential key S. cerevisiae cell wall components, which should be further analyzed to elucidate the biosorption mechanisms involved.

The combined outcomes of the spectroscopic analyses, and the cellular nanomechanical characterization, validate the likelihood of N-linked σ-hole attraction to Pb²⁺ species as a possible mechanism of biosorptive Pb²⁺ retention by the mannoprotein/β-glucan cell wall fraction [Mahmoudi, G. et al., “On the importance of tetrel bonding interactions in lead(II) complexes with (iso)nicotinohydrazide based ligands and several anions”, Dalton Trans. 45, 10708-10716 (2016); Politzer, P., Murray, J. S., Clark, T. & Resnati, G., “The σ-hole revisited”, Phys.Chem.Chem.Phys. 19, 32166-32178 (2017)], leading to supramolecular assemblies that make yeast cells stiffer after biosorption, with potential Pb²⁺ coordination schemes with amide groups having been suggested in the literature [Politzer, P., Murray, J. S., Clark, T. & Resnati, G., “The σ-hole revisited”, Phys.Chem.Chem.Phys. 19, 32166-32178 (2017); Battistuzzi, G., Borsari, M., Menabue, L. & Saladini, M., “Amide Group Coordination to the Pb²⁺ Ion”, Inorg. Chem. 35, 4239-4247 (1996]. These findings open new experimental pathways for approaching the challenging task of biosorption investigation at the ppb scale.

Exploiting yeast as a biosorbent can be practically feasible and economically attractive. S. cerevisiae can be easily cultivated in large quantities, while having various beneficial industrial applications, e.g., in the food, beverage, therapeutics and biofuel production industries [Parapouli, M., Vasileiadis, A., Afendra, A. S. & Hatziloukas, E., “Saccharomyces cerevisiae and its industrial applications”, AIMS Microbiol. 6, 1-31 (2020)]. Three million tons of yeast are used annually by the global fermentation industry [LESAFFRE, “The yeast economy”, Explore Yeast. https://www.exploreyeast.com/article/the-yeast-economy (2021)], while the yeast market is expected to grow by 35% in the next 5 years [Statista, “Value of the yeast product market worldwide from 2017 to 2026”, https://www.statista.com/statistics/728147/global-yeast-product-market-size/(2021)]. However, the sugars and proteins needed for yeast growth are derived from food crops and sources that could be used for human nutrition. Therefore, to avoid compromising food security, residual or surplus yeast from fermentation industries would be a more sustainable option for the large-scale application of this water purification approach. Indeed, surplus yeast is currently produced in huge volumes and is an extremely underutilized low-value resource, not suitable as a human dietary supplement due to high levels of nucleic acids [Jaeger, A., Arendt, E. K., Zannini, E. & Sahin, A. W., “Brewer's Spent Yeast (BSY), an Underutilized Brewing By-Product. Fermentation 6, 123 (2020)].

The present invention compares favorably to many of the highly sophisticated synthetic biology and advanced nanomaterials approaches that have also been examined as candidates for heavy metal removal from water [Yang, J. et al., “Nanomaterials for the Removal of Heavy Metals from Wastewater”, Nanomaterials 9, 424 (2019); Wang, C. et al., “Uranium In Situ Electrolytic Deposition with a Reusable Functional Graphene-Foam Electrode”, Adv. Mater. 2102633 (2021)]. Applying such a low-value resource to remove trace contaminants from water could also result in waste reduction as yeast cells are biodegradable. Moreover, potential desorption processes would allow for heavy metals reclamation, enhancing the application of circular economy models.

Further Materials and Methods

Glassware cleaning. All the glassware used in biosorption experiments was autoclaved for 15 minutes at 121° C. (Tuttnauer Brinkmann 3870E autoclave), then rinsed three times with Type I ultrapure water, with a resistivity of 18.2 MQ cm at 25° C. and Total Organic Carbon (TOC) value <5 ppb (Milli-Q Direct 8 Water Purification System, Millipore Sigma, USA). Phosphate-free detergent, suitable for trace heavy metals analyses, was used to wash the glassware (Liquinox detergent, Alconox, USA), which was then rinsed 3 times with ultrapure water and soaked in a 20% nitric acid (HNO₃) bath for at least 24 hours. The HNO₃ bath was 69% HNO₃(ARISTAR PLUS, VWR Chemicals BDH, VWR, USA) and ultrapure water. After soaking in the 20% HNO₃, flasks were rinsed 3 times with ultrapure water prior to use.

Yeast strain, culture & OD600 culture density measurements. The S. cerevisiae Meyen ex E. C. Hansen MYA-796 strain was used (ATCC, Virginia, USA). YM agar and broth media (ATCC 200 YM Medium, ATCC, Virginia, USA) were used for the yeast cultures. Yeast cells were incubated (Multitron Standard incubator shaker, INFORS HT, USA) at 30° C. and 200 rpm in 2 L Erlenmeyer flasks with shallow medium content (100-200 mL). Discrete time-point OD measurements were performed using 2mL non-frosted cuvettes (VWR Standard Spectrophotometer Cuvettes, VWR, USA) and a tabletop, ultraviolet-visible spectrophotometer measuring at 600 nm (NanoPhotometer NP80, Implen, Munich, Germany). The ATCC 200 YM Medium (agar and broth) was 3 g yeast extract, 3 g malt extract, 10 g dextrose, 5 g peptone and 20 g agar per liter of distilled water.

Biomass harvesting, washing & lyophilization. Yeast cells were harvested by centrifugation at 1 g/2000 rpm for 10 min (Allegra X15-R Centrifuge, USA) and washed by two successive suspensions and centrifugations with ultrapure water. To decide the number of washes required to remove medium residues and metabolites from cells, supernatant samples after each wash were analyzed using HPLC (Aminex HPX-87H Column, Bio-Rad, USA). Harvested, washed cells were kept at −30° C. for 24 h and then inserted in the freeze dryer (Freezone 6 Liter Manifold, Labconco, USA). Each lyophilization cycle (temperature <−40° C., pressure <0.371 mbar) lasted for at least 50 h. Lyophilized cells (powder form) were stored in a desiccator containing silica gel. Freeze-dried biomass was weighted using an analytical balance of 0.1 mg precision and resolution (AB104-S analytical balance, Mettler Toledo, USA).

Aqueous solutions preparation. Type I ultrapure water was spiked with lead(II) nitrate (Pb(NO₃)₂) (Sigma-Aldrich, Millipore Sigma, USA) to achieve solutions with initial Pb²⁺ concentrations of up to 1000 ppb. The initial solution pH was adjusted to the required values by using 69% HNO3 (AMSTAR PLUS, VWR Chemicals BDH, VWR, USA) and 0.1M sodium hydroxide (NaOH) (Carolina Biological, USA). The pH of aqueous solutions was measured using a glass-body electrode suitable for ion-weak samples (InLab Pure Pro-ISM, Mettler Toledo, USA).

Biosorption experiments. All biosorption experiments were conducted in batch contact environments using 2 L Erlenmeyer flasks with lyophilized yeast cells added to 200 mL of Pb2+ containing aqueous solutions. Flasks were incubated at 200 rpm and 25° C. After the required contact time, yeast biomass was separated from the aqueous solutions by centrifugation at 1 g/2000 rpm for 10 min (Allegra X15-R Centrifuge, USA). The supernatants were analyzed using ICP-MS (7900 ICP-MS system, Agilent, USA) to measure the residual Pb²⁺ concentrations following standard operating procedures (Pb²⁺ calibration standards and Bismuth internal standard, Agilent, USA). All experiments were carried out in triplicates and mean values are reported.

For all experiments, a control sample of just ultrapure water spiked with Pb(NO₃)₂ was measured to act as a reference for the initial Pb²⁺ concentration, C₀, in the solution. Pb²⁺ removal measurements were calculated by taking the ICP-MS measurements of the supernatants and subtracting from this the reference to determine the quantity of metal adsorbed by yeast biomass. Type I ultrapure water alone was also tested via ICP-MS to make sure that there was no Pb²⁺ present in the aqueous matrix. The amount of Pb2+ uptake from the yeast biomass was calculated using Equation 1.

The tools developed by Wang and Guo [Wang, J. & Guo, X., “Adsorption kinetic models: Physical meanings, applications, and solving methods”, J. Hazard. Mater. 390, 122-156 (2020); Wang, J. & Guo, X., “Adsorption isotherm models: Classification, physical meaning, application and solving method”, Chemosphere 258, 127279 (2020)] were used for solving the nonlinear adsorption kinetic and isotherm models, using the experimental Pb²⁺ biosorption kinetics data, i.e., the q values measured at specific time intervals (i.e., 0 min, 5 min, 15 min, 30 min, 1 h, 2 h, 4 h, 8 h, 24 h) of contact with yeast biomass (m: 0.005 g) in aqueous solutions (0.2 L) with C₀ of 100 ppb, and isotherm data, i.e., the q values at equilibrium measured after 1 h of contact of yeast biomass (m: 0.005 g) with aqueous solutions (0.2 L) of different initial Pb2+ concentrations (C₀: 20, 40, 100, 200, 300, 500, 700 and 1000 ppb), respectively.

Biosorption experiments and analyses using chitin were conducted following the same steps, procedures, and instruments described above, using chitin instead of yeast biomass (Chitin from shrimp shells, Sigma-Aldrich, Millipore Sigma, USA).

SEM sample preparation and analysis. After biosorption, yeast biomass was harvested via centrifugation at 1 g/2000 rpm for 10 mins (Allegra X15-R Centrifuge, USA) and lyophilized, following the procedures described above. Samples of lyophilized yeast biomass (before and after Pb²⁺ biosorption) were coated in a 20 nm layer of gold (150TES Sputter Coater, EMS, USA), mounted onto carbon double-sided tape, and imaged with an SEM, using secondary electron detection at 10 kV beam voltage and varying magnifications (FlexSEM 1000, Hitachi, Tokyo, Japan).

TEM sample preparation and analysis. Harvested yeast cells were fixed with 2% glutaraldehyde and 2.5% paraformaldehyde in 100mM sodium cacodylate buffer (EMS, USA) for 1 h at 4° C. Then cells were washed twice with 100mM sodium cacodylate buffer, each time re-suspending them by vortexing, storing them at 4° C. for 15 mins, and centrifuging them for 10 mins at 1 g/2000 rpm. Then cells were resuspended in 100mM sodium cacodylate buffer and stayed overnight at 4° C. Pelleted cells were post-fixed for 30 min at 4° C. with 1% osmium tetroxide in 0.1M imidazole of pH 7.5 (EMS, USA). Osmium post-fixation was followed by washing the cells three times with 100mM sodium cacodylate buffer, storing them each time for 15 mins at room temperature. The cells were then washed three times with 0.05M maleate buffer of pH 5.15 (EMS, USA), storing them each time for 15 mins at room temperature. The cells were then stained in 2% uranyl acetate (EMS, USA) overnight, in the dark at room temperature. Stained cells were washed three times with 0.05M maleate buffer of pH 5.15, storing them each time for 15 mins at room temperature. Then, the cells were serially dehydrated using 30%, 35%, 50%, 70%, 90%, 95%, and 100% ethanol solutions. Dehydrated cells were embedded in EMBED-812 resin (EMS, USA). Sections were cut using an ultramicrotome (EM UC7, Leica, Wetzlar, Germany) and a Diatome diamond knife at a thickness setting of 50 nm. The sections were examined using a TEM (Tecnai G2 Spirit TWIN, FEI, USA) at 120 kV.

Measurement of yeast cell wall dimensions. Yeast cell walls were measured using Image J software, taking the average measurements of 15 control and 15 Pb²⁺⁻ exposed cells.

ATR-FTIR analysis. Samples of lyophilized yeast biomass (before and after Pb²⁺ biosorption) were put on the ATR sampling accessory of an FTIR spectrometer (Nicolet iS50 FTIR Spectrometer, Thermo Fisher Scientific, USA). The measured wavenumber range was 400-4000 cm⁻¹, and for all spectra 32 scans were recorded and averaged with a resolution of 4 cm⁻¹ for each spectrum. All the handling material and the ATR sampling surface were cleaned with analytical grade isopropanol and rubbed dry with clean paper before contact with the host material.

XPS analysis. Lyophilized yeast cells were taken with a spatula and loaded on a stainless steel XPS stage (5mm width). All samples were analyzed in the same XPS run (spot size: 400 μm) (K-Alpha XPS system, Thermo Fisher Scientific, USA). Survey scans used a pass energy of 50 eV, while the core scans used a pass energy of 1.5 keV and were energy-calibrated using the C—C bond energy at 284.6 eV. The three peaks observed in the C 1 s spectrum were fitted to identify changes in the C bonds due to Pb²⁺ biosorption. Data processing and peak fitting was done using the Thermo Avantage software and the Powel fitting algorithm. All the handling materials and the polyacetal surface were cleaned with analytical grade isopropanol and rubbed dry with clean paper before contact with the host material.

AFM sample preparation and analysis. Lyophilized yeast biomass (following incubation with or without Pb²⁺ at 200 rpm and 25° C. for 1 h) was harvested via centrifugation at 1 g for 5 mins and adhered to plasma cleaned and CellTak (Corning Inc, USA) adhesive-treated plastic 50×9 mm petri dishes, as described in Gibbs, E., Hsu, J., Barth, K. & Goss, J. W., “Characterization of the nanomechanical properties of the fission yeast (Schizosaccharomyces pombe) cell surface by atomic force microscopy. Yeast 38, 480-492 (2021). AFM force analysis was conducted in liquid (AFM tests were done in hydrated cells) with an MFP-3D AFM (Asylum Research, USA), using spherical colloidal tip CP-PNP-Au-A probes (NanoAndMore, USA) with a sphere diameter of 2 μm and a nominal spring constant of 0.08 N/m. Cell surface indentation was limited to <10% of the hydrated cell wall thickness with a 1.98 μm/s approach velocity. Cellular spring constants (kcell) were calculated using a two-spring model as described in Gibbs et al. and Volle, C. B., Ferguson, M. A., Aidala, K. E., Spain, E. M. & Núñez, M. E., “Quantitative Changes in the Elasticity and Adhesive Properties of Escherichia coli ZK1056 Prey Cells During Predation by Bdellovibrio bacteriovorus”, 109J. Langmuir 24, 8102-8110 (2008).

While preferred embodiments of the invention are disclosed herein, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

What is claimed is:

1. A method for removing micropollutants from an aqueous solution, comprising:
adding a biomass of biomaterial to the solution, the biomaterial comprising at least one biological organism selected for its affinity for biosorption of at least one micropollutant that is present in the solution in a concentration level of at or below parts-per-billion;
controlling the pH and temperature of the aqueous solution to be within a pH range and a temperature range suitable for biosorption of the at least one micropollutant from the solution by the biomaterial; and
maintaining agitation and contact between the biomass and the solution for a time suitable for biosorption of the at least one micropollutant from the solution by the biomaterial.

2. The method of claim 1, wherein the amount of biomass added to the solution is calculated according to the amount of solution, the concentration of the micropollutant in the solution, and the total amount of the micropollutant that can be adsorbed by a particular quantity of the biomaterial.

3. The method of claim 1, wherein the biomaterial comprises at least one yeast.

4. The method of claim 3, wherein the at least one yeast is Saccharomyces cerevisiae.

5. The method of claim 1, wherein the pH range is pH3 to pH7.

6. The method of claim 5, wherein the pH is 5.

7. The method of claim 1, further comprising adjusting the pH of the solution to account for a change in pH of the solution when the biomass is added to the solution.

8. The method of claim 1, wherein the micropollutant is organic.

9. The method of claim 1, wherein the micropollutant is inorganic.

10. The method of claim 9, wherein the inorganic micropollutant comprises at least one heavy metal.

11. The method of claim 10, wherein the at least one heavy metal is lead.

12. The method of claim 1, wherein the biomaterial is active.

13. The method of claim 1, wherein the biomaterial is inactive.

14. A biomaterial for removing a micropollutant from an aqueous solution, comprising:
a biomass of at least one biological organism, the biological organism being selected for its particular affinity for adsorption of a micropollutant that is present in the solution in a concentration level of at or below parts-per-billion, the biological organism having been incubated, harvested, washed, and lyophilized under conditions designed to maximize the ability of the biological organism to adsorb the micropollutant,
wherein the amount of biomass in the biomaterial is calculated according to the amount of solution, the concentration of the micropollutant in the solution, and the total amount of the micropollutant that can be adsorbed by a particular quantity of the biological organism at a prespecified pH, temperature, agitation, and contact time.

15. The biomaterial of claim 14, wherein the biological organism comprises at least one yeast.

16. The biomaterial of claim 15, wherein the at least one yeast is Saccharomyces cerevisiae.

17. The biomaterial of claim 14, wherein the biological organism is further selected for its ability to adsorb the micropollutant at the pH of the aqueous solution or within a pH range to which the aqueous solution can be adjusted.

18. The biomaterial of claim 17, wherein the pH range is pH3 to pH7.

19. The biomaterial of claim 18, wherein the pH is 5.

20. The biomaterial of claim 14, wherein the micropollutant is organic.

21. The biomaterial of claim 14, wherein the micropollutant is inorganic.

22. The biomaterial of claim 21, wherein the at least one inorganic micropollutant comprises at least one heavy metal.

23. The biomaterial of claim 22, wherein the at least one heavy metal is lead.

24. The biomaterial of claim 14, wherein the biological organism is active.

25. The biomaterial of claim 14, wherein the biological organism is inactive.
